(19) United States
(12) Patent Application Publication
(10) Pub. No.: US 2024/0425907 A1
(43) Pub. Date: Dec. 26, 2024

(54) IN-SITU SEQUENCING FOR SPATIAL MULTIOMICS APPLICATIONS

(71) Applicant: ILLUMINA, INC., San Diego, CA (US)

(72) Inventors: Niall Gormley, Cambridge (GB); Anurag Agrawal, San Diego, CA (US); Mathieu Lessard-Viger, San Diego, CA (US); Jonathan Boutell, Bishops Stortford (GB); Eli Carrami, Cambridge (GB)

(21) Appl. No.: 18/749,217

(22) Filed: Jun. 20, 2024

Related U.S. Application Data
(60) Provisional application No. 63/509,177, filed on Jun. 20, 2023.

(51) Int. Cl. C12Q 1/6841; C12Q 1/6874

(52) U.S. Cl. CPC C12Q 1/6841; C12Q 1/6874

(57) ABSTRACT

The present disclosure is generally directed to strategies for spatial multiomics via target nucleic acid capture and amplification.

### CROSS-REFERENCE TO RELATED APPLICATIONS

This application claims the priority benefit under 35 U.S.C. § 119 (e) of U.S. Provisional Application No. 63/509,177, filed Jun. 20, 2023, which is incorporated herein by reference in its entirety.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “IP-2485-P_SeqListing.xml”, which was created on Jun. 12, 2024 and is 9,488 bytes in size. The subject matter of the Sequence Listing is incorporated herein in its entirety by reference.

### BACKGROUND

The ability to determine the identity and location of target analytes in a biological sample is highly desirable. Most of the in situ-based spatial transcriptomics approaches, however, require custom purpose-built instrumentation. Moreover, in situ methods are usually targeted panels and hence de novo mapping using those approaches is not possible.

Ex situ approaches, on the other hand, require barcoding on the capture surface to eventually map the readout back to the original location. These barcoding designs are difficult to achieve.

### SUMMARY

The present disclosure solves the problem of the requirement for a priori knowledge of capture probes by using a general capture probe and readout using sequencing in situ. The barcoding requirement is also solved by the methods described herein, in which detection of a target analyte occurs at the site of capture and therefore barcoding is optional in the methods provided herein.

Accordingly, in some aspects the disclosure provides A method for sequencing nucleic acids, comprising: (a) providing: (i) a plurality of first clustering primers immobilized on a surface, wherein each first clustering primer in the plurality of first clustering primers comprises a first clustering primer sequence; and (ii) a plurality of capture oligonucleotides immobilized on the surface, wherein each capture oligonucleotide in the plurality of capture oligonucleotides comprises (1) the first clustering primer sequence that is immobilized on the surface and (2) a capture nucleotide sequence that is configured to bind to target nucleic acids of a biological sample; (b) contacting the biological sample with the surface, the contacting resulting in hybridization of the target nucleic acids of the biological sample to the capture nucleotide sequences of the plurality of capture oligonucleotides to form hybridized capture oligonucleotides, such that a position of one or more of the target nucleic acids can be correlated with a position in the biological sample; (c) removing the biological sample from the surface; (d) extending the capture nucleotide sequence of the hybridized capture oligonucleotides to form first complementary strands of the target nucleic acids; (e) releasing the target nucleic acids from the surface; (f) ligating an adapter to one or more of the first complementary strands of the target nucleic acids to form ligated first complementary strands, wherein the adapter comprises a binding site for a second clustering primer and a binding site for a sequencing primer; and (g) hybridizing a plurality of second clustering primers to the binding site for the second clustering primer on the ligated first complementary strands to generate hybridized second clustering primers and extending the hybridized second clustering primers to produce second complementary strands that comprise sequences of the target nucleic acids, or portions thereof; (h) separating one or more of the ligated first complementary strands from the second complementary strands to generate separated second complementary strands and hybridizing the separated second complementary strands to a further first clustering primer of the plurality of first clustering primers to generate hybridized first clustering primers; (i) hybridizing further second clustering primers of the plurality of second clustering primers to the binding site for the second clustering primer on the ligated first complementary strands; (j) extending (1) one or more of the hybridized first clustering primers to generate additional first complementary strands, and (2) the further second clustering primers of the plurality of second clustering primers to generate additional second complementary strands; (k) repeating steps (h) to (j) to produce one or more clusters of first complementary strands and second complementary strands; (l) removing the second complementary strands from the surface and hybridizing a plurality of sequencing primers to the binding site for the sequencing primer on the one or more clusters of first complementary strands and sequencing the one or more clusters of first complementary strands to determine a sequence of the one or more clusters of first complementary strands. In some embodiments, the binding site for the sequencing primer of each of the ligated first complementary strands comprises the same nucleotide sequence. In further embodiments, at least two of the ligated first complementary strands comprise different sequences for the sequencing primer binding sites. In some embodiments, the one or more clusters of first complementary strands comprises a plurality of clusters of first complementary strands, and sequencing comprises: (1) hybridizing a first plurality of sequencing primers to a first cluster of first complementary strands having a binding site for the first plurality of sequencing primers, and sequencing the first cluster of first complementary strands; (2) removing the first plurality of sequencing primers from the surface; (3) hybridizing a second plurality of sequencing primers to a second cluster of first complementary strands having a binding site for the second plurality of sequencing primers, and sequencing the second cluster of first complementary strands; and optionally (4) repeating steps (1) to (3) with one or more additional pluralities of sequencing primers. In further aspects, the disclosure provides a method for sequencing nucleic acids, comprising: (a) providing (i) a plurality of first clustering primers immobilized on a surface, wherein each first clustering primer in the plurality of first clustering primers comprises a first clustering primer sequence and (ii) a plurality of capture oligonucleotides immobilized on the surface, wherein each capture oligonucleotide in the plurality of capture oligonucleotides comprises (1) the first clustering primer sequence that is immobilized on the surface and (2) a capture nucleotide sequence that is configured to bind to target nucleic acids of a biological sample; (b) contacting the biological sample with the surface, the contacting resulting in hybridization of the target nucleic acids of the biological sample to the capture nucleotide sequence of the plurality of capture oligonucleotides to form hybridized capture oligonucleotides, such that a position of one or more of the target nucleic acids can be correlated with a position in the biological sample; (c) removing the biological sample from the surface; (d) extending the capture nucleotide sequence of the hybridized capture oligonucleotides to form first complementary strands of the target nucleic acids, wherein the extending comprises addition of a plurality of non-templated nucleotides to the end of the first complementary strands; (e) hybridizing a plurality of template switching oligonucleotides to the plurality of non-templated nucleotides of the first complementary strands such that each of the plurality of template switching oligonucleotides is positioned at the terminus of the target nucleic acids that is distal to the surface, wherein each oligonucleotide in the plurality of template switching oligonucleotides comprises (1) a nucleotide sequence that binds to the plurality of non-templated nucleotides; (2) a sequencing primer sequence; and (3) a second clustering primer sequence; (f) extending the plurality of non-templated nucleotides on the first complementary strands using the template switching oligonucleotides as template, thereby generating a binding site for a second clustering primer and a binding site for a sequencing primer on the first complementary strands; (g) releasing the target nucleic acids from the surface; (h) hybridizing a plurality of second clustering primers to the binding site for the second clustering primer on the first complementary strands to generate hybridized second clustering primers and extending the hybridized second clustering primers to produce second complementary strands that comprise sequences of the target nucleic acids, or portions thereof; (i) separating one or more of the first complementary strands from the second complementary strands to generate separated second complementary strands and hybridizing the separated second complementary strands to a further first clustering primer of the plurality of first clustering primers to generate hybridized first clustering primers; (j) hybridizing further second clustering primers of the plurality of second clustering primers to the binding site for the second clustering primer on the first complementary strands; (k) extending (1) one or more of the hybridized first clustering primers to generate additional first complementary strands, and (2) the further second clustering primers of the plurality of second clustering primers to generate additional second complementary strands; (l) repeating steps (i) to (k) to produce one or more clusters of first complementary strands and second complementary strands; (m) removing the second complementary strands from the surface and hybridizing a plurality of sequencing primers to the binding site for the sequencing primer on the one or more clusters of first complementary strands and sequencing the one or more clusters of first complementary strands to determine a sequence of the one or more clusters of first complementary strands. In some embodiments, the sequencing primer sequence of each of the plurality of template switching oligonucleotides is the same. In further embodiments, at least two of the plurality of template switching oligonucleotides comprise sequencing primer sequences that are different. In some embodiments, the one or more clusters of first complementary strands comprises a plurality of clusters of first complementary strands, and sequencing comprises: (1) hybridizing a first plurality of sequencing primers to a first cluster of first complementary strands having a binding site for the first plurality of sequencing primers, and sequencing the first cluster of first complementary strands; (2) removing the first plurality of sequencing primers from the surface; (3) hybridizing a second plurality of sequencing primers to a second cluster of first complementary strands having a binding site for the second plurality of sequencing primers, and sequencing the second cluster of first complementary strands; and optionally (4) repeating steps (1) to (3) with one or more additional pluralities of sequencing primers. In some embodiments, a method of the disclosure further comprises staining the biological sample after the contacting but before removing the biological sample from the surface. In further embodiments, the staining comprises a tissue stain or an antibody stain. In still further embodiments, the tissue stain is a hematoxylin and eosin stain or a Masson's trichrome stain. In various embodiments, a method of the disclosure further comprises correlating the sequence of the one or more clusters of first complementary strands to a position of the target nucleic acids in the biological sample. In some embodiments, hybridization of the target nucleic acids of the biological sample to the plurality of capture oligonucleotides comprises permeabilizing the biological sample to release the target nucleic acids from the biological sample. In some embodiments, hybridization of the target nucleic acids of the biological sample to the plurality of capture oligonucleotides comprises an electrophoretic transfer of the target nucleic acids from the biological sample to the surface. In further embodiments, the first complementary strand comprises a cDNA molecule. In some embodiments, (a) each sequencing primer in the first plurality of sequencing primers comprises the same nucleotide sequence; (b) each sequencing primer in the second plurality of sequencing primers comprises the same nucleotide sequence; (c) each sequencing primer in each of the one or more additional pluralities of sequencing primers comprises the same nucleotide sequence; and (d) the first plurality of sequencing primers, the second plurality of sequencing primers, and each of the one or more additional pluralities of sequencing primers each comprise different nucleotide sequences. In some embodiments, (a) each sequencing primer in the first plurality of sequencing primers comprises the same nucleotide sequence; (b) each sequencing primer in the second plurality of sequencing primers comprises the same nucleotide sequence; (c) each sequencing primer in each of the one or more additional pluralities of sequencing primers comprises the same nucleotide sequence; and (d) one or more of: the first plurality of sequencing primers, the second plurality of sequencing primers, and each of the one or more additional pluralities of sequencing primers comprise different nucleotide sequences. In some embodiments, the surface is a planar surface or a bead surface. In further embodiments, the planar surface is a flow cell surface. In various embodiments, the ratio of capture oligonucleotides to clustering oligonucleotides on the surface is about 1:10. In further embodiments, the capture oligonucleotides occupy about 0.1% to about 100% of the surface. In some embodiments, the plurality of capture oligonucleotides comprises multiple, different capture nucleotide sequences. In further embodiments, the multiple, different capture nucleotide sequences comprise one or more gene-specific capture sequences, one or more universal capture sequences, or a combination thereof. In some embodiments, the capture nucleotide sequence is a poly-T sequence, a poly-A sequence, a gene-specific capture sequence, or a universal capture sequence. In further embodiments, the universal capture sequence is a random nucleotide sequence or a non-self complementary semi-random sequence. In various embodiments, the target nucleic acids are mRNA, gDNA, rRNA, tRNA, or a combination thereof. In various embodiments, the target nucleic acids are RNA, mRNA, or a combination thereof. In some embodiments, the extending of the capture nucleotide sequence is carried out using a reverse transcriptase. In some embodiments, the target nucleic acids are polyadenylated prior to hybridization of the target nucleic acids to the capture nucleotide sequences. In further embodiments, the target nucleic acids are polyadenylated using a poly(A) polymerase. In some embodiments, the target nucleic acids are polyadenylated using chemical ligation or enzymatic ligation. In various embodiments, the ligating is achieved using chemical ligation or enzymatic ligation. In some embodiments, steps (h) to (j) are repeated through multiple cycles in the presence of a recombinase. In some embodiments, steps (i) to (k) are repeated through multiple cycles in the presence of a recombinase. In further embodiments, the adapter is ligated to the 3′ end of the one or more of the first complementary strands of the target nucleic acids. In some embodiments, releasing the target nucleic acids from the surface is achieved by changing a condition. In further embodiments, the condition is temperature, pH, formamide concentration, or a combination thereof. In some embodiments, the biological sample is a tissue sample. In some embodiments, the method does not comprise use of a spatial barcode.

In some aspects, the disclosure provides a method for obtaining spatial information about target nucleic acids of a biological sample, comprising: (a) providing (i) a plurality of first clustering primers immobilized on a surface, wherein each first clustering primer in the plurality of first clustering primers comprises a first clustering primer sequence and (ii) a plurality of capture oligonucleotides immobilized on the surface, wherein each capture oligonucleotide in the plurality of capture oligonucleotides comprises (1) a first clustering primer sequence that is immobilized on the surface and (2) a capture nucleotide sequence that is configured to bind to the target nucleic acids of the biological sample; (b) contacting the biological sample with the surface, the contacting resulting in hybridization of the target nucleic acids of the biological sample to the capture nucleotide sequences of the plurality of capture oligonucleotides to form hybridized capture oligonucleotides, such that a position of one or more of the target nucleic acids can be correlated with a position in the biological sample; (c) removing the biological sample from the surface; (d) extending the capture nucleotide sequence of the hybridized capture oligonucleotides to form first complementary strands of the target nucleic acids; (e) releasing the target nucleic acids from the surface; (f) ligating an adapter to one or more of the first complementary strands of the target nucleic acids to form ligated first complementary strands, wherein the adapter comprises a binding site for a second clustering primer and a binding site for a sequencing primer; (g) hybridizing a plurality of second clustering primers to the binding site for the second clustering primer on the first complementary strands to generate hybridized second clustering primers and extending the hybridized second clustering primers to produce second complementary strands that comprise sequences of the target nucleic acids, or portions thereof; (h) separating one or more of the ligated first complementary strands from the second complementary strands to generate separated second complementary strands and hybridizing the separated second complementary strands to a further first clustering primer of the plurality of first clustering primers to generate hybridized first clustering primers; (i) hybridizing further second clustering primers of the plurality of second clustering primers to the binding site for the second clustering primer on the ligated first complementary strands; (j) extending (1) one or more of the hybridized first clustering primers to generate additional first complementary strands, and (2) the further second clustering primers of the plurality of second clustering primers to generate additional second complementary strands; (k) repeating steps (h) to (j) to produce one or more clusters of first complementary strands and second complementary strands; (l) removing the second complementary strands from the surface and hybridizing a plurality of sequencing primers to the binding site for the sequencing primer on the one or more clusters of first complementary strands and sequencing the one or more clusters of first complementary strands to determine a sequence of the one or more clusters of first complementary strands; and (m) correlating the sequence of the one or more clusters of first complementary strands to a position of the target nucleic acids in the biological sample. In some embodiments, the binding site for the sequencing primer of each of the ligated first complementary strands comprises the same nucleotide sequence. In further embodiments, the binding site for the sequencing primer of at least two of the ligated first complementary strands comprises a different nucleotide sequence. In various embodiments, the one or more clusters of first complementary strands comprises a plurality of clusters of first complementary strands, and sequencing comprises: (1) hybridizing a first plurality of sequencing primers to a first cluster of first complementary strands having a binding site for the first plurality of sequencing primers, and sequencing the first cluster of first complementary strands; (2) removing the first plurality of sequencing primers from the surface; (3) hybridizing a second plurality of sequencing primers to a second cluster of first complementary strands having a binding site for the second plurality of sequencing primers, and sequencing the second cluster of first complementary strands; and optionally (4) repeating steps (1) to (3) with one or more additional pluralities of sequencing primers. In further aspects, the disclosure provides a method for obtaining spatial information about target nucleic acids of a biological sample, comprising: (a) providing (i) a plurality of first clustering primers immobilized on a surface, wherein each first clustering primer in the plurality of first clustering primers comprises a first clustering primer sequence and (ii) a plurality of capture oligonucleotides immobilized on the surface, wherein each capture oligonucleotide in the plurality of capture oligonucleotides comprises (1) the first clustering primer sequence that is immobilized on the surface and (2) a capture nucleotide sequence that is configured to bind to the target nucleic acids of the biological sample; (b) contacting the biological sample with the surface, the contacting resulting in hybridization of the target nucleic acids of the biological sample to the capture nucleotide sequence of the plurality of capture oligonucleotides to form hybridized capture oligonucleotides, such that a position of one or more of the target nucleic acids can be correlated with a position in the biological sample; (c) removing the biological sample from the surface; (d) extending the capture nucleotide sequence of the hybridized capture oligonucleotides to form first complementary strands of the target nucleic acids, wherein the extending comprises addition of a plurality of non-templated nucleotides to the end of the first complementary strands; (e) hybridizing a plurality of template switching oligonucleotides to the plurality of non-templated nucleotides of the first complementary strands such that each of the plurality of template switching oligonucleotides is positioned at the terminus of the target nucleic acids that is distal to the surface, wherein each oligonucleotide in the plurality of template switching oligonucleotides comprises (1) a nucleotide sequence that binds to the plurality of non-templated nucleotides; (2) a sequencing primer sequence; and (3) a second clustering primer sequence; (f) extending the plurality of non-templated nucleotides on the first complementary strands using the template switching oligonucleotides as template, thereby generating a binding site for a second clustering primer and a binding site for a sequencing primer on the first complementary strands; (g) releasing the target nucleic acids from the surface; (h) hybridizing a plurality of second clustering primers to the binding site for the second clustering primer on the first complementary strands to generate hybridized second clustering primers and extending the hybridized second clustering primers to produce second complementary strands that comprise sequences of the target nucleic acids, or portions thereof; (i) separating one or more of the first complementary strands from the second complementary strands to generate separated second complementary strands and hybridizing the separated second complementary strands to a further first clustering primer of the plurality of first clustering primers to generate hybridized first clustering primers; (j) hybridizing further second clustering primers of the plurality of second clustering primers to the binding site for the second clustering primer on the first complementary strands; (k) extending (1) one or more of the hybridized first clustering primers to generate additional first complementary strands, and (2) the further second clustering primers of the plurality of second clustering primers to generate additional second complementary strands; (l) repeating steps (i) to (k) to produce one or more clusters of first complementary strands and second complementary strands; (m) removing the second complementary strands from the surface and hybridizing a plurality of sequencing primers to the binding site for the sequencing primer on the one or more clusters of first complementary strands and sequencing the one or more clusters of first complementary strands to determine a sequence of the one or more clusters of first complementary strands; and (n) correlating the sequence of the clusters of first complementary strands to a position of the target nucleic acids in the biological sample. In some embodiments, the sequencing primer sequence of each of the plurality of template switching oligonucleotides is the same. In further embodiments, at least two of the plurality of template switching oligonucleotides comprise sequencing primer sequences that are different. In some embodiments, the one or more clusters of first complementary strands comprises a plurality of clusters of first complementary strands, and sequencing comprises: (1) hybridizing a first plurality of sequencing primers to a first cluster of first complementary strands having a binding site for the first plurality of sequencing primers, and sequencing the first cluster of first complementary strands; (2) removing the first plurality of sequencing primers from the surface; (3) hybridizing a second plurality of sequencing primers to a second cluster of first complementary strands having a binding site for the second plurality of sequencing primers, and sequencing the second cluster of first complementary strands; and optionally (4) repeating steps (1) to (3) with one or more additional pluralities of sequencing primers. In various embodiments, a method of the disclosure further comprises staining the biological sample after the contacting but before removing the biological sample from the surface. In further embodiments, the staining comprises a tissue stain or an antibody stain. In still further embodiments, the tissue stain is a hematoxylin and eosin stain or a Masson's trichrome stain. In some embodiments, hybridization of the target nucleic acids of the biological sample to the plurality of capture oligonucleotides comprises permeabilizing the biological sample to release the target nucleic acids from the biological sample. In further embodiments, hybridization of the target nucleic acids of the biological sample to the plurality of capture oligonucleotides comprises an electrophoretic transfer of the target nucleic acids from the biological sample to the surface. In some embodiments, the first complementary strand comprises a cDNA molecule. In still further aspects, the disclosure provides a method for obtaining spatial information about target nucleic acids of a biological sample, comprising: (a) providing: (I) a plurality of capture oligonucleotides immobilized on a surface, wherein each capture oligonucleotide in the plurality of capture oligonucleotides comprises (i) a first clustering primer sequence that is immobilized on the surface; (ii) a sequencing primer sequence; and (iii) a capture nucleotide sequence that is configured to bind to the target nucleic acids of the biological sample; (II) a plurality of first clustering primers immobilized on the surface, wherein each first clustering primer in the plurality of first clustering primers comprises the first clustering primer sequence; and (III) a plurality of dormant second clustering primers immobilized on the surface, wherein each dormant second clustering primer in the plurality of dormant second clustering primers is blocked at the 3′ end; (b) contacting the biological sample with the surface, the contacting resulting in hybridization of the target nucleic acids of the biological sample to the capture nucleotide sequence of the plurality of capture oligonucleotides to form hybridized capture oligonucleotides, such that a position of one or more of the target nucleic acids can be correlated with a position in the biological sample; (c) removing the biological sample from the surface; (d) extending the capture nucleotide sequence of the hybridized capture oligonucleotides to form first complementary strands of the target nucleic acids; (e) releasing the target nucleic acids from the surface; (f) ligating an adapter to one or more of the first complementary strands of the target nucleic acids to form ligated first complementary strands, wherein the adapter comprises a binding site for a third clustering primer and a binding site for a sequencing primer; and (g) hybridizing a plurality of third clustering primers to the binding site for the third clustering primer on the ligated first complementary strands to generate hybridized third clustering primers and extending the hybridized third clustering primers to produce second complementary strands that comprise sequences of the target nucleic acids, or portions thereof; (h) separating one or more of the second complementary strands and hybridizing the one or more of the second complementary strands to the plurality of first clustering primers immobilized on the surface; (i) providing a plurality of primers that hybridizes to the binding site for the third clustering primer of the ligated first complementary strands; (j) extending the third clustering primer and the primer of step (i) to generate additional complementary strands; (k) repeating steps (h) to (j) to produce a cluster of complementary strands; (l) removing strands not immobilized on the surface; (m) carrying out a first sequencing read to determine a sequence of a region of immobilized strands; (n) removing sequencing products; (o) removing the blocking group from the plurality of dormant second clustering primers to allow hybridization of the 3′ end of strands immobilized on the surface to the dormant second clustering primers; (p) extending the dormant second clustering primers using strands immobilized on the surface as a template; (q) removing the first sequencing read strands; (r) carrying out a second sequencing read to determine a sequence of immobilized strands; and correlating the second sequencing reads to the position of the target nucleic acids in the biological sample, thereby obtaining spatial information about the target nucleic acids of the biological sample. In some embodiments, the surface is a planar surface or a bead surface. In further embodiments, the planar surface is a flow cell surface. In various embodiments, the ratio of capture oligonucleotides to clustering oligonucleotides on the surface is about 1:10. In further embodiments, the capture oligonucleotides occupy about 0.1% to about 100% of the surface. In some embodiments, the plurality of capture oligonucleotides comprises multiple, different capture nucleotide sequences. In further embodiments, the multiple, different capture nucleotide sequences comprise one or more gene-specific capture sequences, one or more universal capture sequences, or a combination thereof. In some embodiments, the capture nucleotide sequence is a poly-T sequence, a poly-A sequence, a gene-specific capture sequence, or a universal capture sequence. In further embodiments, the universal capture sequence is a random nucleotide sequence or a non-self complementary semi-random sequence. In various embodiments, the target nucleic acids are mRNA, gDNA, rRNA, tRNA, or a combination thereof. In various embodiments, the target nucleic acids are RNA, mRNA, or a combination thereof. In some embodiments, the extending of the capture nucleotide sequence is carried out using a reverse transcriptase. In some embodiments, the target nucleic acids are polyadenylated prior to hybridization of the target nucleic acids to the capture nucleotide sequences. In further embodiments, the target nucleic acids are polyadenylated using a poly(A) polymerase. In some embodiments, the target nucleic acids are polyadenylated using chemical ligation or enzymatic ligation. In various embodiments, the ligating is achieved using chemical ligation or enzymatic ligation. In some embodiments, steps (h) to (j) are repeated through multiple cycles in the presence of a recombinase. In some embodiments, steps (i) to (k) are repeated through multiple cycles in the presence of a recombinase. In further embodiments, the adapter is ligated to the 3′ end of the one or more of the first complementary strands of the target nucleic acids. In some embodiments, releasing the target nucleic acids from the surface is achieved by changing a condition. In further embodiments, the condition is temperature, pH, formamide concentration, or a combination thereof. In some embodiments, the biological sample is a tissue sample. In some embodiments, the method does not comprise use of a spatial barcode.

### BRIEF DESCRIPTION OF THE DRAWINGS

FIG. 1 depicts exemplary flow cell lawn primer distribution embodiments for capture and sequencing (single-read).

FIG. 2 depicts an exemplary flow cell lawn primer design to enable paired-end sequencing reads.

FIG. 3 is a general schematic for spatial readout of mRNA in a tissue using non-bridging clustering and sequencing by synthesis (SBS).

FIG. 4 depicts sequential readout of captured mRNA to detect the spatial location of densely packed mRNA transcripts.

FIG. 5 shows a template switching approach to add sequencing primers on cDNA in situ. The reverse transcriptase adds a plurality of non-templated nucleotides to the 3′ end of the cDNA. The non-templated nucleotides can anneal to a template switching oligonucleotide (TSO) with a desired sequence handle (for example, sequencing primers SBS3/4/5 etc. and P5), prompting the reverse transcriptase to switch from the mRNA template to the TSO. The resulting cDNA contains the sequencing primer(s) depending on the choice of the TSO pool.

FIG. 6 depicts an alternate flow cell (FC) design to enable mono-clonal capture of mRNA.

### DETAILED DESCRIPTION

Sequencing by synthesis (SBS) (Illumina Inc., San Diego Calif.) technology is generally a bulk sequencing approach in which the readout represents the “average” signal across cells in a sample without preserving context. Spatial multiomics, however, which is the ability to resolve spatial arrangement of multi-omic signals in their tissue context, is an emerging technique. This technique helps to elucidate cellular states, interactions, and signaling to study tissue function, disease progression, and therapy response (e.g., tumor formation/evolution, drug response, metastasis) among other applications.

The present disclosure provides strategies that enable spatial transcriptomics and multi-omics by leveraging surface chemistry, clustering, and sequencing technologies (e.g., Illumina technologies (Illumina Inc., San Diego Calif.)). For common spatial (ex situ) approaches, the library preparation and sequencing readout occurs in a system that is separate from the capture surface. The present disclosure, however, provides the advantage of using the same surface (e.g., flow cell (FC)) for both capture of target analytes and sequencing readout. Thus, methods provided herein eliminate the need to design and prepare complex barcoded surfaces to eventually map readout results to get spatial location information. The strategies described herein utilize a modular design to enable specific applications.

### TERMS

As used in this specification and the enumerated paragraphs herein, the singular forms “a,” “an,” and “the” include plural reference unless the context clearly dictates otherwise.

“About” and “approximately” shall generally mean an acceptable degree of error for the quantity measured given the nature or precision of the measurements. Exemplary degrees of error are within 20-25 percent (%), for example, within 20 percent, 10 percent, 5 percent, 4 percent, 3 percent, 2 percent, or 1 percent of the stated value or range of values.

As used herein, the term “adapter” refers generally to any linear nucleic acid molecule that can be added to an oligonucleotide of the disclosure. In some embodiments, adapters are copied onto the library molecules using templated polymerase synthesis. In some embodiments, adapters include two reverse complementary oligonucleotides forming a double-stranded structure. In some embodiments, an adapter includes two oligonucleotides that are complementary at one portion and mismatched at another portion, forming a Y-shape or fork-shaped adapter that is double stranded at the complementary portion and has two floppy overhangs at the mismatched portion. In some embodiments, an adapter is a template switch oligonucleotide (TSO) adapter. In various embodiments, an adapter comprises a binding site for a primer. In further embodiments, an adapter comprises a binding site for a P5 primer or a P5′ primer. In some embodiments, an adapter comprises a binding site for a P7 primer or a P7′ primer.

The terms “P5”, “P7”, “P5” (P5 prime), and “P7” (P7 prime) may be used when referring to examples of polynucleotide sequences of primers, e.g., clustering primers, and/or polynucleotide sequences of binding sites for primers. The terms “P5” (P5 prime) and “P7” (P7 prime) refer to the complement of P5 and P7, respectively. It will be understood that any suitable primer can be used in the methods presented herein, and that the use of P5, P5′, P7, and P7′ are exemplary embodiments only. Uses of primers such as P5, P5′, P7, and P7′ or their complements on flow cells are known in the art, as exemplified by the disclosures of WO 2007/010251, WO 2006/064199, WO 2005/065814, WO 2015/106941, WO 1998/044151, and WO 2000/018957, each of which is incorporated herein by reference in its entirety. For example, any suitable forward amplification primer, whether immobilized or in solution, can be useful in the methods presented herein for hybridization to a complementary sequence and amplification of a sequence. Similarly, any suitable reverse amplification primer, whether immobilized or in solution, can be useful in the methods presented herein for hybridization to a complementary sequence and amplification of a sequence. One of skill in the art will understand how to design and use primer sequences that are suitable for capture and/or amplification of nucleic acids as presented herein. In some embodiments, a “first clustering primer” as described herein is a P5 primer. In some embodiments, a “first clustering primer” as described herein is a P7 primer. In some embodiments, a “first clustering primer” as described herein is a P5′ primer. In some embodiments, a “first clustering primer” as described herein is a P7′ primer. In some embodiments, a “second clustering primer” as described herein is a P5 primer. In some embodiments, a “second clustering primer” as described herein is a P7 primer. In some embodiments, a “second clustering primer” as described herein is a P5′ primer. In some embodiments, a “second clustering primer” as described herein is a P7′ primer. In some embodiments, a “third clustering primer” as described herein is a P5 primer. In some embodiments, a “third clustering primer” as described herein is a P7 primer. In some embodiments, a “third clustering primer” as described herein is a P5′ primer. In some embodiments, a “third clustering primer” as described herein is a P7′ primer. In some embodiments, P5 comprises or consists of the polynucleotide sequence 5′ AAT GAT ACG GCG ACC ACC GA 3′ (SEQ ID NO: 1), or a variant thereof. In some embodiments, P5 comprises or consists of the polynucleotide sequence 5′ AAT GAT ACG GCG ACC ACC GAG ATC TAC AC 3′ (SEQ ID NO: 2), or a variant thereof. In some embodiments, P7 comprises or consists of the polynucleotide sequence 5′ CAA GCA GAA GAC GGC ATA CG 3′ (SEQ ID NO. 3), or a variant thereof. In some embodiments, P7 comprises or consists of the polynucleotide sequence 5′ CAA GCA GAA GAC GGC ATA CGA GAT 3′ (SEQ ID NO. 4), or a variant thereof. In some embodiments, P5′ comprises or consists of the polynucleotide sequence 5′ TCG GTG GTC GCC GTA TCA TT 3′ (SEQ ID NO: 5), or a variant thereof. In some embodiments, P5′ comprises or consists of the polynucleotide sequence 5′ GTG TAG ATC TCG GTG GTC GCC GTA TCA TT 3′ (SEQ ID NO: 6), or a variant thereof. In some embodiments, P7′ comprises the polynucleotide sequence 5′ CGT ATG CCG TCT TCT GCT TG 3′ (SEQ ID NO. 7), or a variant thereof. In some embodiments, P7′ comprises or consists of the polynucleotide sequence 5′ ATC TCG TAT GCC GTC TTC TGC TTG 3′ (SEQ ID NO. 8), or a variant thereof. The term “variant” as used herein with reference to any of the sequences recited herein refers to a variant nucleic acid that is substantially identical, i.e., has only some nucleotide sequence variations, for example to the non-variant sequence. In some embodiments, a variant has at least 50%, 55%, 60%, 65%, 70%, 75%, 80%, 85%, 86%, 87%, 88%, 89%, 90%, 91%, 92%, 93%, 94%, 95%, 96%, 97%, 98%, or at least 99% overall nucleotide sequence identity to the non-variant nucleic acid sequence. It will be understood that reference to P5 and P7 herein could refer to different primer sequences. Any suitable primer sequence combinations are encompassed by the present disclosure.

The term “bead” refers to a small body made of a rigid or semi-rigid material. The body can have a shape characterized, for example, as a sphere, oval, microsphere, or other recognized particle shape whether having regular or irregular dimensions. Example materials that are useful for beads include, without limitation, glass; plastic such as acrylic, polystyrene or a copolymer of styrene and another material, polypropylene, polyethylene, polybutylene, polyurethane or polytetrafluoroethylene (TEFLON®, from Chemours); polysaccharides or cross-linked polysaccharides such as agarose or Sepharose; nylon; nitrocellulose; resin; silica or silica-based materials including silicon and modified silicon; carbon-fiber, metal; inorganic glass; optical fiber bundle, or a variety of other polymers. Example beads include, without limitation, controlled pore glass beads, paramagnetic beads, thoria sol, Sepharose beads, nanocrystals and others known in the art as described, for example, in Microsphere Detection Guide from Bangs Laboratories, Fishers Ind. Beads may also be coated with a polymer that has a functional group that can attach to an oligonucleotide.

As used herein, “surface” can refer to a part of a substrate or support structure that is accessible to contact with reagents, beads, or analytes. The surface can be substantially flat or planar. Alternatively, the surface can be rounded or contoured. Example contours that can be included on a surface are wells (e.g., microwells or nanowells), depressions, pillars, ridges, channels or the like. Example materials that can be used as a substrate or support structure include glass such as modified or functionalized glass; plastic such as acrylic, polystyrene or a copolymer of styrene and another material, polypropylene, polyethylene, polybutylene, polyurethane or TEFLON; polysaccharides or cross-linked polysaccharides such as agarose or Sepharose; nylon; nitrocellulose; resin; silica or silica-based materials including silicon and modified silicon, carbon-fibre; metal; inorganic glass; optical fibre bundle, or a variety of other polymers. A single material or mixture of several different materials can form a surface useful in certain examples. In some examples, a surface comprises wells (e.g., microwells or nanowells). In some aspects, the surface comprises an array of wells (e.g., microwells or nanowells) on glass, silicon, plastic or other suitable solid supports with patterned, covalently-linked gel such as poly(N-(5-azidoacetamidylpentyl) acrylamide-coacrylamide) (PAZAM, see, for example, U.S. Pat. App. Pub. No. 2014/0079923 A1, which is incorporated herein by reference). In some embodiments, each nanowell comprises a unique oligonucleotide (e.g., an oligonucleotide with a unique spatial barcode). In some examples, a support structure can include one or more layers. Non-limiting examples of a surface include a bead array, a spotted array, clustered particles arranged on a surface of a chip, a film, a multi-well plate, and a flow cell.

Exemplary flow cells include but are not limited to those used in a nucleic acid sequencing apparatus such as flow cells for the Genome Analyzer®, MiSeq®, NextSeq® or HiSeq® platforms commercialized by Illumina, Inc. (San Diego, Calif.); or for the SOLID™ or Ion Torrent™ sequencing platform commercialized by Life Technologies (Carlsbad, Calif.). Exemplary flow cells and methods for their manufacture and use are also described, for example, in WO 2014/142841 A1; U.S. Pat. App. Pub. No. 2010/0111768 A1 and U.S. Pat. No. 8,951,781, each of which is incorporated herein by reference.

A bead array comprising oligonucleotide barcodes can also be used in a sequencing procedure, such as a sequencing-by-synthesis (SBS) technique. Briefly, SBS can be initiated by contacting the barcodes with one or more labeled nucleotides, DNA polymerase, etc. Those features where a primer is extended using the sequences comprising the barcode as a template will incorporate a labeled nucleotide that can be detected. Optionally, the labeled nucleotides can further include a reversible termination property that terminates further primer extension once a nucleotide has been added to a primer. For example, a nucleotide analog having a reversible terminator moiety can be added to a primer such that subsequent extension cannot occur until a deblocking agent is delivered to remove the moiety. Thus, for embodiments that use reversible termination, a deblocking reagent can be delivered to the flow cell (before or after detection occurs). Washes can be carried out between the various delivery steps. The cycle can then be repeated n times to extend the primer by n nucleotides, thereby detecting a sequence of length n. Exemplary SBS procedures, fluidic systems and detection platforms that can be readily adapted for use with an array produced by the methods of the present disclosure are described, for example, in Bentley et al., Nature 456:53-59 (2008), WO 04/018497; WO 91/06678; WO 07/123744; U.S. Pat. Nos. 7,057,026; 7,329,492; 7,211,414; 7,315,019 or 7,405,281, and US Pat. App. Pub. No. 2008/0108082 A1, each of which is incorporated herein by reference.

As used herein, the term “barcode” is intended to mean a series of nucleotides in an oligonucleotide that can be used to identify the oligonucleotide, a spatial address on a surface, a characteristic of the oligonucleotide, or a manipulation that has been carried out on the oligonucleotide. The barcode can be a naturally occurring nucleotide sequence or a nucleotide sequence that does not occur naturally in the organism from which the barcoded nucleic acid was obtained. A barcode sequence can be unique to a single nucleic acid species in a population or a barcode sequence can be shared by several different nucleic acid species in a population. For example, each nucleic acid capture probe in a population on a substrate for spatial capture of nucleic acids in a biological sample, e.g., a permeabilized biological sample (e.g., tissue sample), a cell suspension, can include different barcode sequences from all other nucleic acid capture probes in the population. Alternatively, each nucleic acid probe in a population can include different barcode sequences from some or most other nucleic acid capture probes in a population. For example, each capture probe in a population can have a barcode that is present for several different capture probes in the population even though the capture probes with the common barcode differ from each other at other sequence regions along their length. In various embodiments, one or more barcode sequences that are used with a biological tissue are not present in the genome, transcriptome or other nucleic acids of the biological specimen. For example, barcode sequences can have less than 80%, 70%, 60%, 50% or 40% sequence identity to the nucleic acid sequences in a particular biological tissue.

As used herein, the term “array” refers to a population of sites that can be differentiated from each other according to relative location. Different molecules that are at different sites of an array can be differentiated from each other according to the locations of the sites in the array. An individual site of an array can include one or more molecules of a particular type. For example, a site can include a single nucleic acid molecule having a particular sequence or a site can include several nucleic acid molecules having the same sequence (and/or complementary sequence, thereof). The sites of an array can be different features located on the same substrate. Exemplary features include without limitation, beads (or other particles) in or on a substrate, droplets, wells in a substrate, projections from a substrate, ridges on a substrate or channels in a substrate. The sites of an array can be separate substrates each bearing a different molecule. Different molecules attached to separate substrates can be identified according to the locations of the substrates on a surface to which the substrates are associated or according to the locations of the substrates in a liquid or gel. Exemplary arrays in which separate substrates are located on a surface include, without limitation, those having beads in wells.

As used herein, a “biological sample” may include one or more biological or chemical substances, such as nucleic acids, oligonucleotides, proteins, cells, tissues, organisms, and/or biologically active chemical compound(s), such as analogs or mimetics of the aforementioned species. As used herein, the term “tissue” is intended to mean an aggregation of cells, and, optionally, intercellular matter. Typically the cells in a tissue are not free floating in solution and instead are attached to each other to form a multicellular structure. Exemplary tissue types include muscle, nerve, epidermal and connective tissues. In some instances, the biological sample may include whole blood, lymphatic fluid, serum, plasma, sweat, tear, saliva, sputum, cerebrospinal fluid, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, fecal samples, liquids containing single or multiple cells, liquids containing organelles, fluidized tissues, fluidized organisms, viruses including viral pathogens, liquids containing multi-celled organisms, biological swabs and biological washes. In further examples, the sample can be derived from an organ, including for example, an organ of the musculoskeletal system such as muscle, bone, tendon or ligament; an organ of the digestive system such as salivary gland, pharynx, esophagus, stomach, small intestine, large intestine, liver, gallbladder or pancreas; an organ of the respiratory system such as larynx, trachea, bronchi, lungs or diaphragm; an organ of the urinary system such as kidney, ureter, bladder or urethra; a reproductive organ such as ovary, fallopian tube, uterus, vagina, placenta, testicle, epididymis, vas deferens, seminal vesicle, prostate, penis or scrotum; an organ of the endocrine system such as pituitary gland, pineal gland, thyroid gland, parathyroid gland, or adrenal gland; an organ of the circulatory system such as heart, artery, vein or capillary; an organ of the lymphatic system such as lymphatic vessel, lymph node, bone marrow, thymus or spleen; an organ of the central nervous system such as brain, brainstem, cerebellum, spinal cord, cranial nerve, or spinal nerve; a sensory organ such as eye, ear, nose, or tongue; or an organ of the integument such as skin, subcutaneous tissue or mammary gland. In various embodiments, the tissue can be derived from a multicellular organism. In some embodiments, a tissue section can be contacted with a surface, for example, by laying the tissue on the surface. The tissue can be freshly excised from an organism or it may have been previously preserved for example by freezing (e.g., fresh frozen tissue), embedding in a material such as paraffin (e.g., formalin fixed paraffin embedded (FFPE) samples), formalin fixation, infiltration, dehydration or the like. Optionally, a tissue section can be attached to a surface, for example, using techniques and compositions described in, for example, U.S. Pat. No. 11,390,912, incorporated by reference herein in its entirety. In some embodiments, a tissue can be permeabilized and the cells of the tissue lysed when the tissue is in contact with a surface. Any of a variety of treatments can be used such as those set forth above in regard to lysing cells. Target proteins and/or nucleic acids that are released from a tissue that is permeabilized can be captured by capture oligonucleotides on the surface. The thickness of a tissue sample or other biological sample that is contacted with a surface in a method set forth herein can be any suitable thickness desired. In representative embodiments, the thickness will be at least 0.1 μm, 0.25 μm, 0.5 μm, 0.75 μm, 1 μm, 5 μm, 10 μm, 50 μm, 100 μm or thicker. Alternatively or additionally, the thickness of a biological sample that is contacted with a surface will be no more than 100 μm, 50μ, 10 μm, 5 μm, 1 μm, 0.5 μm, 0.25 μm, 0.1 μm or thinner.

As used herein, a “capture oligonucleotide” is generally an oligonucleotide comprising a nucleotide sequence capable of hybridizing or otherwise associating with an analyte (e.g., target nucleic acid). In various embodiments, a capture oligonucleotide comprises a clustering primer sequence and a capture nucleotide sequence that is configured to bind to target nucleic acids of a biological sample. In some embodiments, a capture oligonucleotide comprises a clustering primer sequence (e.g., a P5 sequence) and a capture nucleotide sequence (e.g., a poly T sequence), and is between about 30 bases to about 100 bases in length, or between about 30 bases to about 90 bases, or between about 30 bases and 80 bases, or between about 30 bases and 70 bases, or between about 30 bases and 60 bases, or between about 30 bases and 55 bases, or between about 30 bases and 50 bases in length. In further embodiments, a capture oligonucleotide comprises a clustering primer sequence (e.g., a P5 sequence) and a capture nucleotide sequence (e.g., a poly T sequence), and is about 30 bases, 35 bases, 40 bases, 45 bases, 50 bases, 55 bases, 60 bases, 65 bases, 70 bases, 75 bases, 80 bases, 85 bases, 90 bases, 95 bases, or 100 bases in length. The capture nucleotide sequence capable of hybridizing or otherwise associating with an analyte is, for example and without limitation, an aptamer, a universal sequence (e.g., a polyT sequence, a random nucleotide sequence, or a semi-random nucleotide sequence), or a target-specific (e.g., a gene-specific) sequence. In various embodiments, a capture nucleotide sequence (e.g., a poly T nucleotide sequence or a random nucleotide sequence) is, is about, or is at least about 2, 5, 8, 10, 12, 15, 18, 20, 22, 25, 28, 30, 32, 35, 38, 40, or more bases in length. Alternatively or additionally, a capture nucleotide sequence can include less than or equal to about 40, 38, 35, 32, 30, 28, 25, 22, 20, 18, 15, 12, 10, 8, 5, or 2 bases. A capture oligonucleotide can comprise additional elements, including but not limited to a unique molecular identifier (UMI), a spatial barcode, primer sequences to amplify from (e.g., A14-ME), sequences that are used to generate the barcoded features (e.g., a P7 sequence used in clustering and a SBS12 sequence used as the sequencing primer binding site) or a combination thereof.

As used herein, a “semi-random” nucleotide sequence comprises or consists of a partially pre-determined nucleotide sequence combined with a random nucleotide sequence.

As used herein, the term “different”, when used in reference to nucleic acids, means that the nucleic acids have nucleotide sequences that are not the same as each other. Two or more nucleic acids can have nucleotide sequences that are different along their entire length. Alternatively, two or more nucleic acids can have nucleotide sequences that are different along a substantial portion of their length. For example, two or more nucleic acids can have target nucleotide sequence portions that are different for the two or more molecules while also having a universal sequence portion that is the same on the two or more molecules. The term can be similarly applied to proteins which are distinguishable as different from each other based on amino acid sequence differences.

As used herein, the term “clustering oligonucleotide” or “clustering primer” refers to a nucleotide sequence in solution and/or immobilized on a surface that is used for amplifying the template polynucleotides to create identical copies of the same templates (i.e., clusters). Examples of clustering oligonucleotides may include but are not limited to P5 primer, P5′ primer, P7 primer, P7′ primer, P15 primer, P15′ primer, P17′ primer, and P17′ primer as described herein. In some embodiments, the “clustering primer” is also referred to as a “surface primer.”

By “complementary” is meant that the primer has a sequence of nucleotides that can form a double-stranded structure by matching base-pairs with the adaptor or primer sequence or part thereof. By “substantially complementary” is meant that the primer has at least 85%, 90%, 95%, 98%, 99% or 100% overall sequence identical to the complementary sequence.

As used herein, “hybridize” is intended to mean noncovalently associating a first oligonucleotide to a second oligonucleotide along the lengths of those polymers to form a double-stranded “duplex.” For instance, two DNA oligonucleotide strands may associate through complementary base pairing. The strength of the association between the first and second oligonucleotides increases with the complementarity between the sequences of nucleotides within those oligonucleotides. The strength of hybridization between oligonucleotides may be characterized by a temperature of melting (Tm) at which 50% of the duplexes have oligonucleotide strands that disassociate from one another. Oligonucleotides that are “partially” hybridized to one another means that they have sequences that are complementary to one another, but such sequences are hybridized with one another along only a portion of their lengths to form a partial duplex. Oligonucleotides with an “inability” to hybridize include those that are physically separated from one another such that an insufficient number of their bases may contact one another in a manner so as to hybridize with one another.

As used herein the term “analyte” is intended to include any of a variety of moieties that are to be detected, characterized, modified, synthesized, or the like. Exemplary analytes include, but are not limited to, nucleic acids (e.g., DNA, RNA or analogs thereof), proteins, polysaccharides, cells, nuclei, cellular organelles, antibodies, epitopes, receptors, ligands, enzymes (e.g., kinases, phosphatases or polymerases), peptides, small molecule drug candidates, or the like. Thus, in any of the aspects or embodiments of the disclosure an analyte is a “target nucleic acid” that is a RNA molecule, such as a mRNA. An array can include multiple different species from a library of analytes. For example, the species can be different antibodies from an antibody library, nucleic acids having different sequences from a library of nucleic acids, proteins having different structure and/or function from a library of proteins, drug candidates from a combinatorial library of small molecules, etc.

As used herein, a “primer” is a nucleic acid molecule that can hybridize to a target sequence, such as an adapter attached to an oligonucleotide (e.g., a first complementary strand as described herein). As one example, an amplification primer can serve as a starting point for template amplification and cluster generation. As another example, a synthesized nucleic acid (template) strand may include a site to which a primer (e.g., a sequencing primer) can hybridize in order to prime synthesis of a new strand that is complementary to the synthesized nucleic acid strand. Any primer can include any combination of nucleotides or analogs thereof. In some examples, the primer is a single-stranded oligonucleotide or polynucleotide. The primer length can be any number of bases long and can include a variety of non-natural nucleotides. In various embodiments, the sequencing primer is a short strand, ranging from 10 to 60 bases, or from 20 to 40 bases.

As used herein, the term “unique molecular identifier” or “UMI” refers to a molecular tag, either random, non-random, or semi-random, that may be attached to a nucleic acid. When incorporated into a nucleic acid, a UMI can be used to correct for subsequent amplification bias by directly counting unique molecular identifiers (UMIs) that are sequenced after amplification. A UMI can be attached to similar nucleic acids, e.g., adapters, making each nucleic acid unique. UMI's may also be used to uniquely tag individual molecules (e.g., individual mRNA molecules) in a sample (e.g., individual mRNA molecules in a tissue sample, cell sample, or sample library).

As used herein, the term “universal sequence” refers to a series of nucleotides that is common to two or more nucleic acid molecules even if the molecules also have regions of sequence that differ from each other. A universal sequence that is present in different members of a collection of molecules can allow capture of multiple different nucleic acids using a population of universal capture nucleic acids that are complementary to the universal sequence. Similarly, a universal sequence present in different members of a collection of molecules can allow the replication or amplification of multiple different nucleic acids using a population of universal primers that are complementary to the universal sequence. Thus, a universal capture nucleic acid or a universal primer includes a sequence that can hybridize specifically to a universal sequence. Target nucleic acid molecules may be modified to attach universal adapters, for example, at one or both ends of the different target sequences. Universal capture oligonucleotides are applicable for interrogating a plurality of different oligonucleotides without necessarily distinguishing the different species whereas target-specific capture sequences are applicable for distinguishing the different species. A non-limiting example of a universal sequence is a polyT nucleotide sequence.

As used herein, the term “plurality” is intended to mean a population of two or more members, which may all be the same or two or more members may be different. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above example ranges. Accordingly, the definition of the term is intended to include all integer values greater than two. An upper limit of a plurality may be set, for example, by the theoretical diversity of bead types in an array.

As used herein, a “semi-random” nucleotide sequence comprises or consists of a partially pre-determined nucleotide sequence combined with a random nucleotide sequence.

### Oligonucleotides

An oligonucleotide is a polymer comprised of nucleotides. Oligonucleotides of the disclosure may be of any length and include, in various embodiments, DNA oligonucleotides, RNA oligonucleotides, analogs thereof, or a combination thereof. In any aspects or embodiments described herein, an oligonucleotide is single-stranded, double-stranded, or partially double-stranded.

Nucleotides may include naturally occurring nucleotides and functional analogs thereof. Examples of functional analogs are those that are capable of hybridizing to a nucleic acid in a sequence specific fashion or capable of being used as a template for replication of a particular nucleotide sequence. Naturally occurring nucleotides generally have a backbone containing phosphodiester bonds. An analog structure can have an alternate backbone linkage including any of a variety known in the art. Naturally occurring nucleotides generally have a deoxyribose sugar (e.g., found in DNA) or a ribose sugar (e.g., found in RNA). An analog structure can have an alternate sugar moiety including any of a variety known in the art. Nucleotides can include native or non-native bases. A native DNA can include one or more of adenine, thymine, cytosine and/or guanine, and a native RNA can include one or more of adenine, uracil, cytosine and/or guanine. Any non-native base may be used, such as a locked nucleic acid (LNA) and a bridged nucleic acid (BNA). Example modified nucleotides include inosine, xathanine, hypoxathanine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

Oligonucleotides contemplated by the disclosure also include those having at least one modified internucleotide linkage. In some embodiments, the oligonucleotide is all or in part a peptide nucleic acid. Other modified internucleoside linkages include at least one phosphorothioate linkage. Still other modified oligonucleotides include those comprising one or more universal bases. “Universal base” refers to molecules capable of substituting for binding to any one of A, C, G, T and U in nucleic acids by forming hydrogen bonds without significant structure destabilization. Examples of universal bases include but are not limited to 5′-nitroindole-2′-deoxyriboside, 3-nitropyrrole, inosine and hypoxanthine.

In various aspects, an oligonucleotide of the disclosure, or a modified form thereof, is generally about 5 nucleotides to about 150 nucleotides in length. In further embodiments, an oligonucleotide of the disclosure is about 5 to about 125 nucleotides in length, about 5 to about 100 nucleotides in length, about 5 to about 90 nucleotides in length, about 5 to about 50 nucleotides in length, about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, about 10 to about 150 nucleotides in length, about 10 to about 125 nucleotides in length, about 10 to about 100 nucleotides in length, about 10 to about 90 about 10 to about 50 nucleotides in length, about 10 to about 45 nucleotides in length, about 10 to about 40 nucleotides in length, about 10 to about 35 nucleotides in length, about 10 to about 30 nucleotides in length, about 10 to about 25 nucleotides in length, about 10 to about 20 nucleotides in length, about 10 to about 15 nucleotides in length, and all oligonucleotides intermediate in length of the sizes specifically disclosed to the extent that the oligonucleotide is able to achieve the desired result. Accordingly, in various embodiments, an oligonucleotide of the disclosure is or is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 or more nucleotides in length. In further embodiments, an oligonucleotide of the disclosure is less than 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or more nucleotides in length. In various embodiments, the length of an oligonucleotide (such as a primer) of the disclosure is between about 5 base pairs (bp) and 40 bp, or between about 5 bp and 35 bp, or between about 5 bp and 30 bp, or between about 10 bp and 35 bp, or between about 10 bp and 30 bp, or between about 20 bp and 40 bp, or between about 20 bp and 35 bp, or between about 20 bp and 30 bp, or between about 9 and 20 bp or between about 5 and 15 bp, or between about 9 and 15 bp in length. In some embodiments, the length of an oligonucleotide (such as a primer) of the disclosure is about 10 bp, 13 bp, 15 bp, 20 bp, 25 bp, 30 bp, 35 bp, or 40 bp. As described herein, in various embodiments the oligonucleotide may be a P5 primer, a P5′ primer, a P7 primer, or a P7′ primer.

As used herein, the term “poly T” or “poly A,” when used in reference to a nucleic acid sequence (e.g., a capture nucleotide sequence), is intended to mean a series of two or more thiamine (T) or adenine (A) bases, respectively. A poly T or poly A can include at least about 2, 5, 8, 10, 12, 15, 18, 20, 22, 25, 28, 30, 32, 35, 38, 40, or more of the T or A bases, respectively. Alternatively or additionally, a poly T or poly A can include at most about 40, 38, 35, 32, 30, 28, 25, 22, 20, 18, 15, 12, 10, 8, 5, or 2 of the T or A bases, respectively. In some embodiments, the disclosure contemplates use of a “polyTVN” sequence, which is a poly T sequence followed by a V (any base but a T) and an N. The polyTVN sequence is used, in some embodiments, to bias reverse transcription to the base of the poly A tail on the mRNA molecule.

As used herein, the term “immobilized” refers to the state of two things being joined, fastened, adhered, attached, connected, or bound to each other. For example, an analyte, such as a nucleic acid, can be immobilized on a material, such as a bead, gel, or surface, by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions and hydrophobic interactions. In various embodiments, covalent attachment can be used, but all that is required is that the oligonucleotides remain stationary or attached to a surface under conditions in which it is intended to use the surface, for example, in applications requiring nucleic acid capture, amplification, and/or sequencing. Oligonucleotides to be used as capture oligonucleotides can be immobilized such that a 3′-end is available for enzymatic extension and at least a portion of the sequence is capable of hybridizing to a complementary sequence.

Exemplary covalent linkages include, for example, those that result from the use of click chemistry techniques. Exemplary non-covalent linkages include, but are not limited to, non-specific interactions (e.g., hydrogen bonding, ionic bonding, van der Waals interactions etc.) or specific interactions (e.g., affinity interactions, receptor-ligand interactions, antibody-epitope interactions, avidin-biotin interactions, streptavidin-biotin interactions, lectin-carbohydrate interactions, etc.). Exemplary linkages are set forth in U.S. Pat. Nos. 6,737,236; 7,259,258; 7,375,234 and 7,427,678; and US Pat. Pub. No. 2011/0059865 A1, each of which is incorporated herein by reference.

As used herein, the term “extend,” when used in reference to a nucleic acid, is intended to mean addition of at least one nucleotide to the nucleic acid. In particular embodiments one or more nucleotides can be added to the 3′ end of a nucleic acid, for example, via polymerase catalysis (e.g. DNA polymerase, RNA polymerase or reverse transcriptase). Chemical or enzymatic methods can be used to add one or more nucleotide to the 3′ or 5′ end of a nucleic acid. An extension reaction, in which nucleotides are added to the 3′ end of an oligonucleotide (e.g., a primer) is performed in the presence of a polymerase, such as a DNA or RNA polymerase. In some embodiments, the polymerase is a non-thermostable isothermal strand displacement polymerase. Suitable non-thermostable strand displacement polymerases according to the present disclosure can be found, for example, through New England BioLabs, Inc. and include phi29, Bsu, Klenow, DNA Polymerase I (E. coli), and Therminator. In some embodiments, the extension reaction is carried out by recombinase polymerase amplification (RPA). RPA comprises three core enzymes—a recombinase, a single-stranded DNA binding protein (SSB) and a strand-displacing polymerase. As described in Daher et al. (Rana K Daher, Gale Stewart, Maurice Boissinot, Michel G Bergeron, Recombinase Polymerase Amplification for Diagnostic Applications, Clinical Chemistry, Volume 62, Issue 7, 1 Jul. 2016). One or more oligonucleotides can be added to the 3′ or 5′ end of a nucleic acid, for example, via chemical or enzymatic (e.g., ligase catalysis) methods. A nucleic acid can be extended in a template directed manner, whereby the product of extension is complementary to a template nucleic acid that is hybridized to the nucleic acid that is extended.

### Methods

The present disclosure is generally directed to methods of in situ sequencing for, e.g., spatial multiomics applications. Accordingly, in various aspects the disclosure provides methods for spatially capturing target nucleic acids of a tissue sample. Put another way, in some aspects the disclosure provides methods for spatially preserving the location of target nucleic acids in a biological sample (e.g., tissue sample). In general, technology disclosure herein provides several advantages. First, the technology of the disclosure provides the ability to capture target analytes (e.g., mRNA or other analytes for multi-omic approaches, proteomics using aptamer detection) on a surface (e.g., an Illumina flow cell (Illumina Inc., San Diego Calif.)) followed by sequencing readout using an appropriate (e.g., Illumina Inc., San Diego Calif.) sequencing infrastructure. Such technology allows untargeted spatial detection of mRNA/analytes to allow for de-novo mapping of signals in the tissue context. Second, technology of the disclosure provides the ability to modify the approach to targeted/untargeted detection to avoid molecular crowding for applications requiring detection of densely packed transcripts.

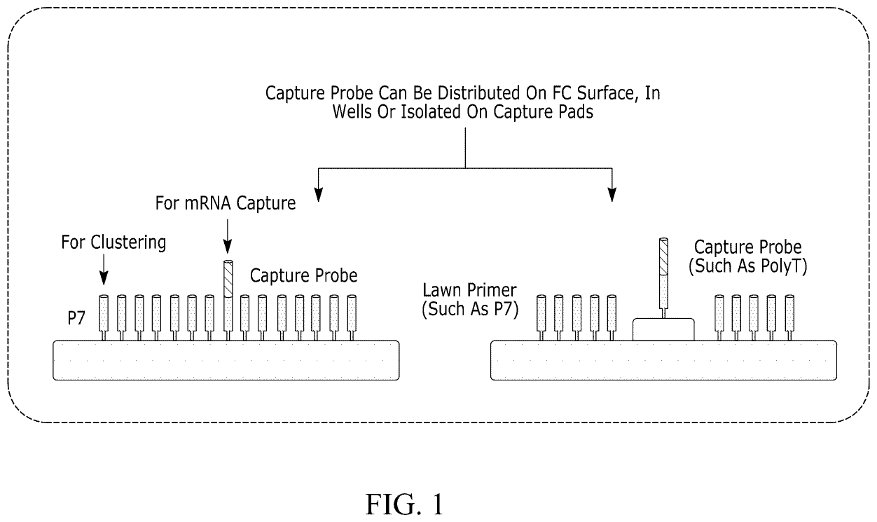

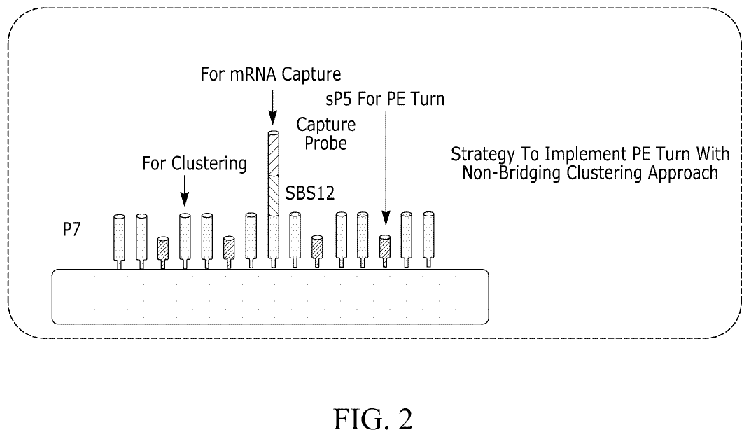

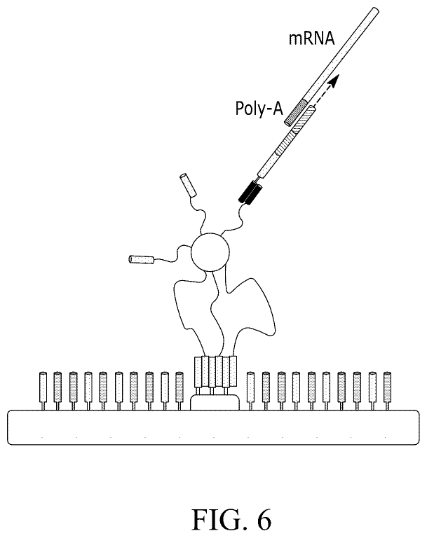

For the approaches disclosed herein, the design for the flow cell (FC) that is used for both capture as well as sequencing can be tuned based on desired applications and required quality metrics (see, e.g., FIG. 1 and FIG. 2). For applications requiring only a single read the lawn primers can be distributed in FC features such as isolated capture pads and nano-wells or uniformly (e.g., a “random” FC) (see, e.g., FIG. 1). In some embodiments, distributed primers are useful for uniform clusters, localized transcript detections, and for enabling monoclonal seeding technologies (see, e.g., FIG. 6).

Although standard (e.g., Illumina Inc., San Diego Calif.) single-read capabilities of 150 cycles should be sufficient for most spatial applications including the whole transcriptome sequencing, the primer design can be changed to enable paired end reads if needed for a specific approach (see, e.g., FIG. 2). FIG. 2 depicts the lawn primers for embodiments that enable paired end (PE) turn using non-bridge clustering. In some embodiments, a short blocked adaptor (e.g., P5 (also known as “dormant P5”) is added to achieve the non-bridging-based PE turn, as well as a decoding spatial barcode segment (e.g., SBS12) on the capture probe to enable performing a second read. Thus, in some embodiments, sequencing methods comprise paired-end (PE) re-synthesis. In various embodiments, PE re-synthesis is achieved using “blocked” or “dormant” primers immobilized on a surface. These primers do not participate in cluster generation but only in re-synthesis prior to sequencing. Thus, in some embodiments, the primer immobilized on a surface is blocked at the 3′ end, wherein the block is removed prior to re-synthesis. For example, in some embodiments the block is removed following generation of the cluster. In this way the primer immobilized on the surface can be considered dormant until the sequencing step. The 3′ block may be a phosphate group or another reversible blocking group. In some embodiments, the surface is treated with a phosphatase to remove the block. In various embodiments, the ratio of clustering primer:dormant primer is 5:1, 4:1, 3:1, 2:1, 1:1, 1:2, 1:3, 1:4 or 1:5. In some embodiments, the ratio of clustering primer:dormant primer is 2:1, 1:1, or 1:2.

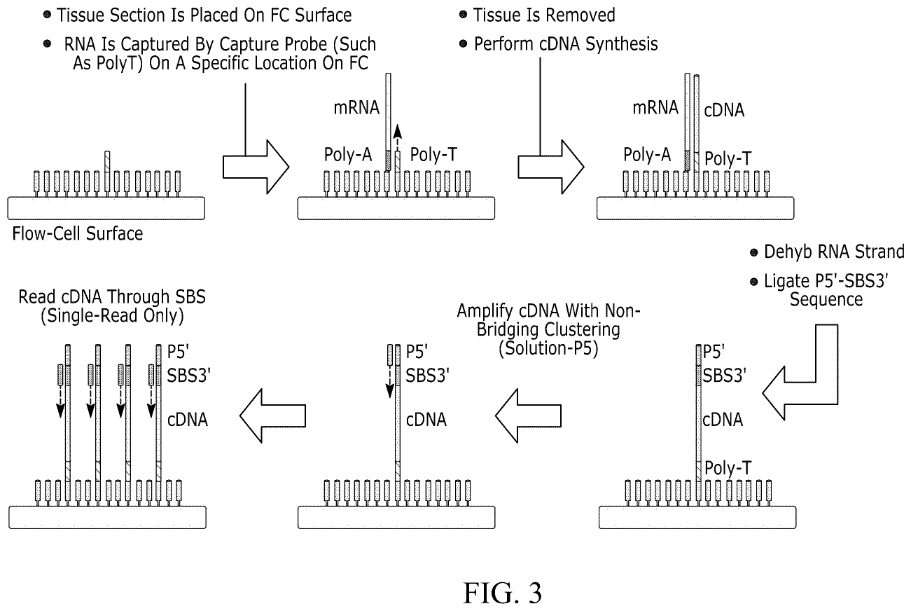

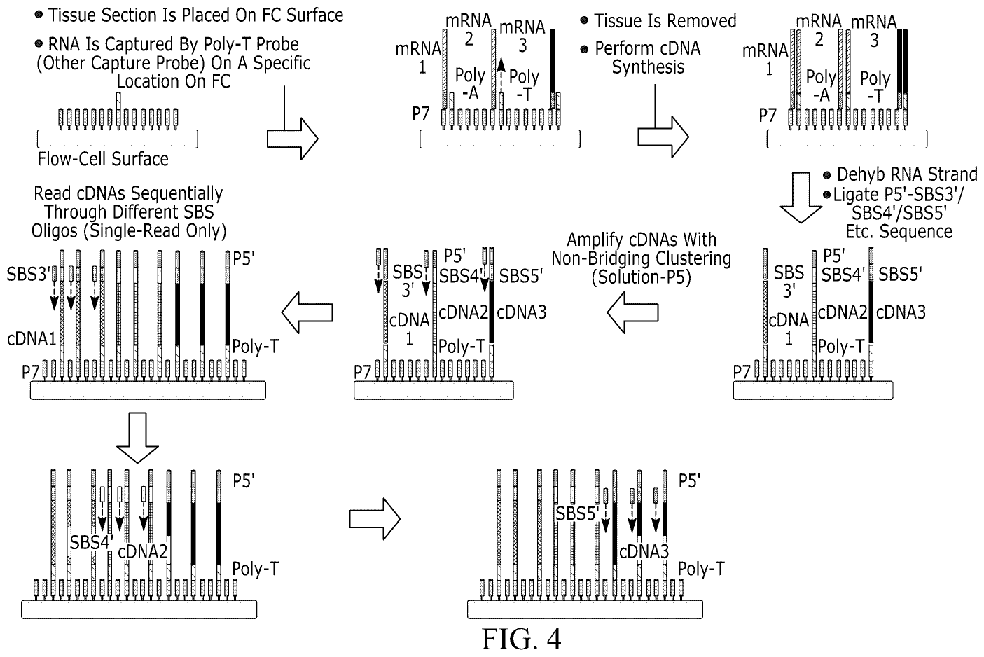

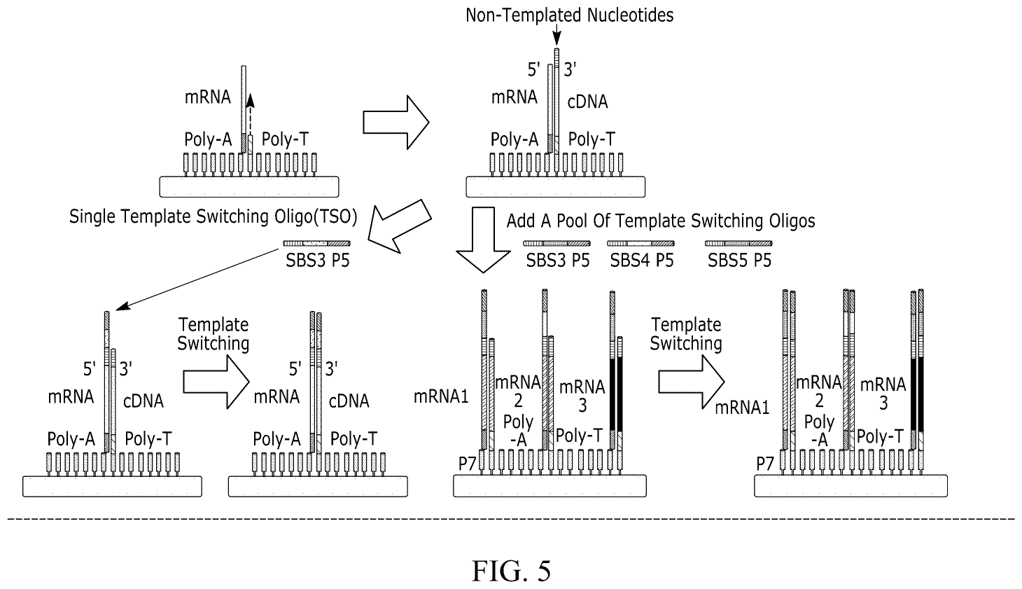

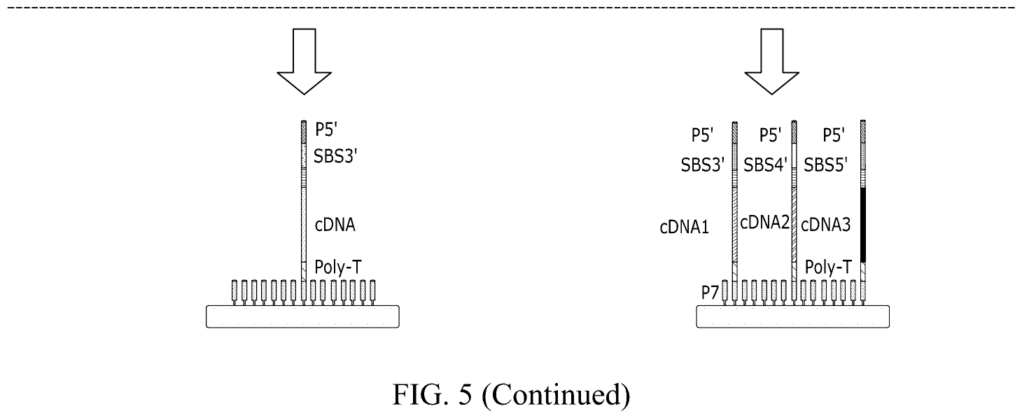

With a FC designed as shown in FIG. 1, a general scheme (see, e.g., FIG. 3) may be used to acquire spatial readout from target analytes (e.g., target nucleic acids such as mRNA transcripts) captured in a biological sample (e.g., tissue sample) (see, e.g., FIG. 3) or additionally any other analytes that leverage this approach. In various embodiments, methods of the disclosure comprise placing a biological section (e.g., tissue section) on a FC which can then be imaged with common staining procedures (e.g., H&E staining). The section is then processed and permeabilized to release target analytes (e.g., mRNA transcripts) to the FC. The target analytes are then captured in situ on the FC with a capture oligonucleotide (e.g., an oligonucleotide comprising or consisting of a polyT nucleotide sequence may be used with fresh frozen tissue or with formalin fixed paraffin embedded (FFPE) samples). After performing cDNA synthesis, ligation of a primer set (e.g., P5′-SBS3′ or other primer set for clustering/sequencing) can be performed in situ to allow for clustering and sequencing with a temporary fluidic fixture to close the FC for those purposes. Such a ligation can also be achieved using a template switch oligonucleotide approach (see, e.g., FIG. 5). A consideration for this method is the resolution of densely packed mRNA transcripts in certain regions of the desired biological sample (e.g., tissue). The mRNA expression levels can be variable depending on the cell types and if multiple types of mRNAs are captured close to each other then optical overcrowding can make the resolution of such mRNAs difficult. The disclosure contemplates that in some embodiments, a solution to this potential issue is a variation to the above-described approach. For example, the process can be modified to have separation between sequencing signals through sequential reading (see, e.g., FIG. 4). The difference in this approach is ligation of multiple sequencing primer sets on a particular capture pad. Sequencing can be performed on a mRNA transcript sequentially by only hybridizing one type of sequencing complementary primer at a time. This allows only one type of primer set in the cluster to light up at a time, hence making the resolution easier. The ligation (or other alternate methods to add SBS primer oligonucleotides) of the SBS primer oligonucleotides on the cDNA strands can be designed for specific attachment to cDNA strands of interest to achieve targeted sequencing or can be kept random to achieve signal separation for untargeted sequencing.

In further embodiments, another approach to solve the close capture of mRNA transcripts is to de-couple capture probe and clustering probes by consolidating seeding primers (capture oligonucleotides) to a small area (see, e.g., FIG. 6). This approach minimizes the capture oligonucleotide sites and mono-clonality of the clusters is achieved.

Accordingly, in some aspects the disclosure provides a method for sequencing nucleic acids, comprising: (a) providing: (i) a plurality of first clustering primers immobilized on a surface, wherein each first clustering primer in the plurality of first clustering primers comprises a first clustering primer sequence; and (ii) a plurality of capture oligonucleotides immobilized on the surface, wherein each capture oligonucleotide in the plurality of capture oligonucleotides comprises (1) the first clustering primer sequence that is immobilized on the surface and (2) a capture nucleotide sequence that is configured to bind to target nucleic acids of a biological sample; (b) contacting the biological sample with the surface, the contacting resulting in hybridization of the target nucleic acids of the biological sample to the capture nucleotide sequences of the plurality of capture oligonucleotides to form hybridized capture oligonucleotides, such that a position of one or more of the target nucleic acids can be correlated with a position in the biological sample; (c) removing the biological sample from the surface; (d) extending the capture nucleotide sequence of the hybridized capture oligonucleotides to form first complementary strands of the target nucleic acids; (e) releasing the target nucleic acids from the surface; (f) ligating an adapter to one or more of the first complementary strands of the target nucleic acids to form ligated first complementary strands, wherein the adapter comprises a binding site for a second clustering primer and a binding site for a sequencing primer; and (g) hybridizing a plurality of second clustering primers to the binding site for the second clustering primer on the ligated first complementary strands to generate hybridized second clustering primers and extending the hybridized second clustering primers to produce second complementary strands that comprise sequences of the target nucleic acids, or portions thereof; (h) separating one or more of the ligated first complementary strands from the second complementary strands to generate separated second complementary strands and hybridizing the separated second complementary strands to a further first clustering primer of the plurality of first clustering primers to generate hybridized first clustering primers; (i) hybridizing further second clustering primers of the plurality of second clustering primers to the binding site for the second clustering primer on the ligated first complementary strands; (j) extending (1) one or more of the hybridized first clustering primers to generate additional first complementary strands, and (2) the further second clustering primers of the plurality of second clustering primers to generate additional second complementary strands; (k) repeating steps (h) to (j) to produce one or more clusters of first complementary strands and second complementary strands; (l) removing the second complementary strands from the surface and hybridizing a plurality of sequencing primers to the binding site for the sequencing primer on the one or more clusters of first complementary strands and sequencing the one or more clusters of first complementary strands to determine a sequence of the one or more clusters of first complementary strands. In various embodiments, steps (h) to (j) are repeated through multiple cycles in the presence of a recombinase. In some embodiments, the recombinase is an isothermal recombinase and the multiple cycles are performed at about 38° C. for about 1 hour. In some embodiments, the binding site for the sequencing primer of each of the ligated first complementary strands comprises the same nucleotide sequence. In some embodiments, at least two of the ligated first complementary strands comprise different sequences for the sequencing primer binding sites. In further embodiments, the one or more clusters of first complementary strands comprises a plurality of clusters of first complementary strands, and sequencing comprises: (1) hybridizing a first plurality of sequencing primers to a first cluster of first complementary strands having a binding site for the first plurality of sequencing primers, and sequencing the first cluster of first complementary strands; (2) removing the first plurality of sequencing primers from the surface; (3) hybridizing a second plurality of sequencing primers to a second cluster of first complementary strands having a binding site for the second plurality of sequencing primers, and sequencing the second cluster of first complementary strands; and optionally (4) repeating steps (1) to (3) with one or more additional pluralities of sequencing primers. In further aspects, the disclosure provides a method for sequencing nucleic acids, comprising: (a) providing (i) a plurality of first clustering primers immobilized on a surface, wherein each first clustering primer in the plurality of first clustering primers comprises a first clustering primer sequence and (ii) a plurality of capture oligonucleotides immobilized on the surface, wherein each capture oligonucleotide in the plurality of capture oligonucleotides comprises (1) the first clustering primer sequence that is immobilized on the surface and (2) a capture nucleotide sequence that is configured to bind to target nucleic acids of a biological sample; (b) contacting the biological sample with the surface, the contacting resulting in hybridization of the target nucleic acids of the biological sample to the capture nucleotide sequence of the plurality of capture oligonucleotides to form hybridized capture oligonucleotides, such that a position of one or more of the target nucleic acids can be correlated with a position in the biological sample; (c) removing the biological sample from the surface; (d) extending the capture nucleotide sequence of the hybridized capture oligonucleotides to form first complementary strands of the target nucleic acids, wherein the extending comprises addition of a plurality of non-templated nucleotides to the end of the first complementary strands; (e) hybridizing a plurality of template switching oligonucleotides to the plurality of non-templated nucleotides of the first complementary strands such that each of the plurality of template switching oligonucleotides is positioned at the terminus of the target nucleic acids that is distal to the surface, wherein each oligonucleotide in the plurality of template switching oligonucleotides comprises (1) a nucleotide sequence that binds to the plurality of non-templated nucleotides; (2) a sequencing primer sequence; and (3) a second clustering primer sequence; (f) extending the plurality of non-templated nucleotides on the first complementary strands using the template switching oligonucleotides as template, thereby generating a binding site for a second clustering primer and a binding site for a sequencing primer on the first complementary strands; (g) releasing the target nucleic acids from the surface; (h) hybridizing a plurality of second clustering primers to the binding site for the second clustering primer on the first complementary strands to generate hybridized second clustering primers and extending the hybridized second clustering primers to produce second complementary strands that comprise sequences of the target nucleic acids, or portions thereof; (i) separating one or more of the first complementary strands from the second complementary strands to generate separated second complementary strands and hybridizing the separated second complementary strands to a further first clustering primer of the plurality of first clustering primers to generate hybridized first clustering primers; (j) hybridizing further second clustering primers of the plurality of second clustering primers to the binding site for the second clustering primer on the first complementary strands; (k) extending (1) one or more of the hybridized first clustering primers to generate additional first complementary strands, and (2) the further second clustering primers of the plurality of second clustering primers to generate additional second complementary strands; (l) repeating steps (i) to (k) to produce one or more clusters of first complementary strands and second complementary strands; (m) removing the second complementary strands from the surface and hybridizing a plurality of sequencing primers to the binding site for the sequencing primer on the one or more clusters of first complementary strands and sequencing the one or more clusters of first complementary strands to determine a sequence of the one or more clusters of first complementary strands. In various embodiments, steps (i) to (k) are repeated through multiple cycles in the presence of a recombinase. In some embodiments, the recombinase is an isothermal recombinase and the multiple cycles are performed at about 38° C. for about 1 hour. In some embodiments, the sequencing primer sequence of each of the plurality of template switching oligonucleotides is the same. In some embodiments, at least two of the plurality of template switching oligonucleotides comprise sequencing primer sequences that are different. In further embodiments, the one or more clusters of first complementary strands comprises a plurality of clusters of first complementary strands, and sequencing comprises: (1) hybridizing a first plurality of sequencing primers to a first cluster of first complementary strands having a binding site for the first plurality of sequencing primers, and sequencing the first cluster of first complementary strands; (2) removing the first plurality of sequencing primers from the surface; (3) hybridizing a second plurality of sequencing primers to a second cluster of first complementary strands having a binding site for the second plurality of sequencing primers, and sequencing the second cluster of first complementary strands; and optionally (4) repeating steps (1) to (3) with one or more additional pluralities of sequencing primers. In some embodiments, methods of the disclosure further comprise staining the biological sample after the contacting but before removing the biological sample from the surface. In further embodiments, the staining comprises a tissue stain or an antibody stain. In still further embodiments, the tissue stain is a hematoxylin and eosin stain or a Masson's trichrome stain. In some embodiments, methods of the disclosure further comprise correlating the sequence of the one or more clusters of first complementary strands to a position of the target nucleic acids in the biological sample. In some embodiments, hybridization of the target nucleic acids of the biological sample to the plurality of capture oligonucleotides comprises permeabilizing the biological sample to release the target nucleic acids from the biological sample. In various embodiments, hybridization of the target nucleic acids of the biological sample to the plurality of capture oligonucleotides comprises an electrophoretic transfer of the target nucleic acids from the biological sample to the surface. In some embodiments, the first complementary strand comprises a cDNA molecule. In some embodiments, (a) each sequencing primer in the first plurality of sequencing primers comprises the same nucleotide sequence; (b) each sequencing primer in the second plurality of sequencing primers comprises the same nucleotide sequence; (c) each sequencing primer in each of the one or more additional pluralities of sequencing primers comprises the same nucleotide sequence; and (d) the first plurality of sequencing primers, the second plurality of sequencing primers, and each of the one or more additional pluralities of sequencing primers each comprise different nucleotide sequences. In some embodiments, (a) each sequencing primer in the first plurality of sequencing primers comprises the same nucleotide sequence; (b) each sequencing primer in the second plurality of sequencing primers comprises the same nucleotide sequence; (c) each sequencing primer in each of the one or more additional pluralities of sequencing primers comprises the same nucleotide sequence; and (d) one or more of: the first plurality of sequencing primers, the second plurality of sequencing primers, and each of the one or more additional pluralities of sequencing primers comprise different nucleotide sequences.

In further aspects, the disclosure provides a method for obtaining spatial information about target nucleic acids of a biological sample, comprising: (a) providing (i) a plurality of first clustering primers immobilized on a surface, wherein each first clustering primer in the plurality of first clustering primers comprises a first clustering primer sequence and (ii) a plurality of capture oligonucleotides immobilized on the surface, wherein each capture oligonucleotide in the plurality of capture oligonucleotides comprises (1) a first clustering primer sequence that is immobilized on the surface and (2) a capture nucleotide sequence that is configured to bind to the target nucleic acids of the biological sample; (b) contacting the biological sample with the surface, the contacting resulting in hybridization of the target nucleic acids of the biological sample to the capture nucleotide sequences of the plurality of capture oligonucleotides to form hybridized capture oligonucleotides, such that a position of one or more of the target nucleic acids can be correlated with a position in the biological sample; (c) removing the biological sample from the surface; (d) extending the capture nucleotide sequence of the hybridized capture oligonucleotides to form first complementary strands of the target nucleic acids; (e) releasing the target nucleic acids from the surface; (f) ligating an adapter to one or more of the first complementary strands of the target nucleic acids to form ligated first complementary strands, wherein the adapter comprises a binding site for a second clustering primer and a binding site for a sequencing primer; (g) hybridizing a plurality of second clustering primers to the binding site for the second clustering primer on the first complementary strands to generate hybridized second clustering primers and extending the hybridized second clustering primers to produce second complementary strands that comprise sequences of the target nucleic acids, or portions thereof; (h) separating one or more of the ligated first complementary strands from the second complementary strands to generate separated second complementary strands and hybridizing the separated second complementary strands to a further first clustering primer of the plurality of first clustering primers to generate hybridized first clustering primers; (i) hybridizing further second clustering primers of the plurality of second clustering primers to the binding site for the second clustering primer on the ligated first complementary strands; (j) extending (1) one or more of the hybridized first clustering primers to generate additional first complementary strands, and (2) the further second clustering primers of the plurality of second clustering primers to generate additional second complementary strands; (k) repeating steps (h) to (j) to produce one or more clusters of first complementary strands and second complementary strands; (l) removing the second complementary strands from the surface and hybridizing a plurality of sequencing primers to the binding site for the sequencing primer on the one or more clusters of first complementary strands and sequencing the one or more clusters of first complementary strands to determine a sequence of the one or more clusters of first complementary strands; and (m) correlating the sequence of the one or more clusters of first complementary strands to a position of the target nucleic acids in the biological sample. In various embodiments, steps (h) to (j) are repeated through multiple cycles in the presence of a recombinase. In some embodiments, the recombinase is an isothermal recombinase and the multiple cycles are performed at about 38° C. for about 1 hour. In some embodiments, the binding site for the sequencing primer of each of the ligated first complementary strands comprises the same nucleotide sequence. In some embodiments, the binding site for the sequencing primer of at least two of the ligated first complementary strands comprises a different nucleotide sequence. In further embodiments, the one or more clusters of first complementary strands comprises a plurality of clusters of first complementary strands, and sequencing comprises: (1) hybridizing a first plurality of sequencing primers to a first cluster of first complementary strands having a binding site for the first plurality of sequencing primers, and sequencing the first cluster of first complementary strands; (2) removing the first plurality of sequencing primers from the surface; (3) hybridizing a second plurality of sequencing primers to a second cluster of first complementary strands having a binding site for the second plurality of sequencing primers, and sequencing the second cluster of first complementary strands; and optionally (4) repeating steps (1) to (3) with one or more additional pluralities of sequencing primers. In further aspects, the disclosure provides a method for obtaining spatial information about target nucleic acids of a biological sample, comprising: (a) providing (i) a plurality of first clustering primers immobilized on a surface, wherein each first clustering primer in the plurality of first clustering primers comprises a first clustering primer sequence and (ii) a plurality of capture oligonucleotides immobilized on the surface, wherein each capture oligonucleotide in the plurality of capture oligonucleotides comprises (1) the first clustering primer sequence that is immobilized on the surface and (2) a capture nucleotide sequence that is configured to bind to the target nucleic acids of the biological sample; (b) contacting the biological sample with the surface, the contacting resulting in hybridization of the target nucleic acids of the biological sample to the capture nucleotide sequence of the plurality of capture oligonucleotides to form hybridized capture oligonucleotides, such that a position of one or more of the target nucleic acids can be correlated with a position in the biological sample; (c) removing the biological sample from the surface; (d) extending the capture nucleotide sequence of the hybridized capture oligonucleotides to form first complementary strands of the target nucleic acids, wherein the extending comprises addition of a plurality of non-templated nucleotides to the end of the first complementary strands; (e) hybridizing a plurality of template switching oligonucleotides to the plurality of non-templated nucleotides of the first complementary strands such that each of the plurality of template switching oligonucleotides is positioned at the terminus of the target nucleic acids that is distal to the surface, wherein each oligonucleotide in the plurality of template switching oligonucleotides comprises (1) a nucleotide sequence that binds to the plurality of non-templated nucleotides; (2) a sequencing primer sequence; and (3) a second clustering primer sequence; (f) extending the plurality of non-templated nucleotides on the first complementary strands using the template switching oligonucleotides as template, thereby generating a binding site for a second clustering primer and a binding site for a sequencing primer on the first complementary strands; (g) releasing the target nucleic acids from the surface; (h) hybridizing a plurality of second clustering primers to the binding site for the second clustering primer on the first complementary strands to generate hybridized second clustering primers and extending the hybridized second clustering primers to produce second complementary strands that comprise sequences of the target nucleic acids, or portions thereof; (i) separating one or more of the first complementary strands from the second complementary strands to generate separated second complementary strands and hybridizing the separated second complementary strands to a further first clustering primer of the plurality of first clustering primers to generate hybridized first clustering primers; (j) hybridizing further second clustering primers of the plurality of second clustering primers to the binding site for the second clustering primer on the first complementary strands; (k) extending (1) one or more of the hybridized first clustering primers to generate additional first complementary strands, and (2) the further second clustering primers of the plurality of second clustering primers to generate additional second complementary strands; (l) repeating steps (i) to (k) to produce one or more clusters of first complementary strands and second complementary strands; (m) removing the second complementary strands from the surface and hybridizing a plurality of sequencing primers to the binding site for the sequencing primer on the one or more clusters of first complementary strands and sequencing the one or more clusters of first complementary strands to determine a sequence of the one or more clusters of first complementary strands; and (n) correlating the sequence of the clusters of first complementary strands to a position of the target nucleic acids in the biological sample. In various embodiments, steps (i) to (k) are repeated through multiple cycles in the presence of a recombinase. In some embodiments, the recombinase is an isothermal recombinase and the multiple cycles are performed at about 38° C. for about 1 hour. In some embodiments, the sequencing primer sequence of each of the plurality of template switching oligonucleotides is the same. In some embodiments, at least two of the plurality of template switching oligonucleotides comprise sequencing primer sequences that are different. In further embodiments, the one or more clusters of first complementary strands comprises a plurality of clusters of first complementary strands, and sequencing comprises: (1) hybridizing a first plurality of sequencing primers to a first cluster of first complementary strands having a binding site for the first plurality of sequencing primers, and sequencing the first cluster of first complementary strands; (2) removing the first plurality of sequencing primers from the surface; (3) hybridizing a second plurality of sequencing primers to a second cluster of first complementary strands having a binding site for the second plurality of sequencing primers, and sequencing the second cluster of first complementary strands; and optionally (4) repeating steps (1) to (3) with one or more additional pluralities of sequencing primers. In some embodiments, the methods further comprise staining the biological sample after the contacting but before removing the biological sample from the surface. In further embodiments, the staining comprises a tissue stain or an antibody stain. In still further embodiments, the tissue stain is a hematoxylin and eosin stain or a Masson's trichrome stain. In some embodiments, hybridization of the target nucleic acids of the biological sample to the plurality of capture oligonucleotides comprises permeabilizing the biological sample to release the target nucleic acids from the biological sample. In some embodiments, hybridization of the target nucleic acids of the biological sample to the plurality of capture oligonucleotides comprises an electrophoretic transfer of the target nucleic acids from the biological sample to the surface. In various embodiments, the first complementary strand comprises a cDNA molecule.

In further aspects, the disclosure provides a method for obtaining spatial information about target nucleic acids of a biological sample, comprising: (a) providing: (I) a plurality of capture oligonucleotides immobilized on a surface, wherein each capture oligonucleotide in the plurality of capture oligonucleotides comprises (i) a first clustering primer sequence that is immobilized on the surface; (ii) a sequencing primer sequence; and (iii) a capture nucleotide sequence that is configured to bind to the target nucleic acids of the biological sample; (II) a plurality of first clustering primers immobilized on the surface, wherein each first clustering primer in the plurality of first clustering primers comprises the first clustering primer sequence; and (III) a plurality of dormant second clustering primers immobilized on the surface, wherein each dormant second clustering primer in the plurality of dormant second clustering primers is blocked at the 3′ end; (b) contacting the biological sample with the surface, the contacting resulting in hybridization of the target nucleic acids of the biological sample to the capture nucleotide sequence of the plurality of capture oligonucleotides to form hybridized capture oligonucleotides, such that a position of one or more of the target nucleic acids can be correlated with a position in the biological sample; (c) removing the biological sample from the surface; (d) extending the capture nucleotide sequence of the hybridized capture oligonucleotides to form first complementary strands of the target nucleic acids; (e) releasing the target nucleic acids from the surface; (f) ligating an adapter to one or more of the first complementary strands of the target nucleic acids to form ligated first complementary strands, wherein the adapter comprises a binding site for a third clustering primer and a binding site for a sequencing primer; and (g) hybridizing a plurality of third clustering primers to the binding site for the third clustering primer on the ligated first complementary strands to generate hybridized third clustering primers and extending the hybridized third clustering primers to produce second complementary strands that comprise sequences of the target nucleic acids, or portions thereof; (h) separating one or more of the second complementary strands and hybridizing the one or more of the second complementary strands to the plurality of first clustering primers immobilized on the surface; (i) providing a plurality of primers that hybridizes to the binding site for the third clustering primer of the ligated first complementary strands; (j) extending the third clustering primer and the primer of step (i) to generate additional complementary strands; (k) repeating steps (h) to (j) to produce a cluster of complementary strands; (l) removing strands not immobilized on the surface; (m) carrying out a first sequencing read to determine a sequence of a region of immobilized strands; (n) removing sequencing products; (o) removing the blocking group from the plurality of dormant second clustering primers to allow hybridization of the 3′ end of strands immobilized on the surface to the dormant second clustering primers; (p) extending the dormant second clustering primers using strands immobilized on the surface as a template; (q) removing the first sequencing read strands; (r) carrying out a second sequencing read to determine a sequence of immobilized strands; and correlating the second sequencing reads to the position of the target nucleic acids in the biological sample, thereby obtaining spatial information about the target nucleic acids of the biological sample. In various embodiments, steps (h) to (j) are repeated through multiple cycles in the presence of a recombinase. In some embodiments, the recombinase is an isothermal recombinase and the multiple cycles are performed at about 38° C. for about 1 hour.

In some embodiments, the surface is a planar surface or a bead surface. In further embodiments, the planar surface is a flow cell surface. In various embodiments, the ratio of capture oligonucleotides to clustering oligonucleotides on the surface is about 1:100. In further embodiments, the ratio of capture oligonucleotides to clustering oligonucleotides on the surface is or is about 1:100 to about 100:1. In further embodiments, the ratio of capture oligonucleotides to clustering oligonucleotides on the surface is or is about 1:100, 1:50, 1:25, 1:10, 1:5, 1:2, 1:1, 2:1, 5:1, 10:1, 25:1, 50:1, or 100:1. In various examples, the capture oligonucleotides occupy about 0.1% to about 100%, about 0.1% to about 90%, about 0.1% to about 80%, about 0.1% to about 70%, about 0.1% to about 60%, about 0.1% to about 50%, about 0.1% to about 40%, about 0.1% to about 30%, about 0.1% to about 20%, about 0.1% to about 10%, about 0.1% to about 5%, about 10% to about 100%, about 10% to about 90%, about 10% to about 80%, about 10% to about 70%, about 10% to about 60%, about 10% to about 50%, about 10% to about 40%, about 10% to about 30%, or about 10% to about 20% of the surface. In further embodiments, the capture oligonucleotides occupy about, at least about, or less than about 0.1%, 0.5%, 1%, 5%, 10%, 20%, 30%, 40%, 50%, 60%, 70%, 80%, 90%, or 100% of the surface.

In various embodiments, the primer that hybridizes to the binding site for the second clustering primer (or third clustering primer, in some embodiments) on the ligated first complementary strands is used at a concentration in the range of 0.1 μM to 100 μM, 1 μM to 100 μM or 3 μM to 75 μM or 5 to 50 μM. In some embodiments, the primer is used at a concentration of 0.25 μM, 0.5 μM or 1.1 μM or 2.2 μM. In still further embodiments, the primer is used at a concentration of 1 μM 5 μM, 10 μM, 25 μM or 50 μM. In various embodiments, such a primer is a P5 primer, a P5′ primer, a P7 primer, or a P7′ primer. In various embodiments, the template-switching oligonucleotide is used at a concentration in the range of 0.1 μM to 100 μM, 1 μM to 100 μM or 3 μM to 75 μM or 5 to 50 μM. In further embodiments, the template-switching oligonucleotide is used at a concentration of 0.25 μM, 0.5 μM or 1.1 μM or 2.2 μM. In some embodiments, the template-switching oligonucleotide is used at a concentration of 1 μM 5 μM, 10 μM, 25 μM or 50 μM.

In some embodiments, the capture nucleotide sequences of the plurality of capture oligonucleotides comprise multiple, different capture nucleotide sequences. In further embodiments, the multiple, different capture nucleotide sequences comprise one or more target (e.g., gene)-specific capture sequences, one or more universal capture sequences, or a combination thereof. In various embodiments, the capture nucleotide sequence is a poly-T sequence, a poly-A sequence, a gene-specific capture sequence, or a universal capture sequence. In still further embodiments, the universal capture sequence is a random nucleotide sequence or a non-self complementary semi-random sequence. In various embodiments, the target nucleic acids are mRNA, gDNA, rRNA, tRNA, or a combination thereof. In various embodiments, the target nucleic acids are RNA, mRNA, or a combination thereof. In further embodiments, the target nucleic acids are mRNA. In various embodiments, the extending of the capture nucleotide sequence is carried out using a reverse transcriptase. In some embodiments, the target nucleic acids are polyadenylated prior to hybridization of the target nucleic acids to the capture nucleotide sequences. In further embodiments, the target nucleic acids are polyadenylated using a poly(A) polymerase. In some embodiments, the target nucleic acids are polyadenylated using chemical ligation or enzymatic ligation. In further embodiments, the ligating is achieved using chemical ligation or enzymatic ligation. In some embodiments, the clustering adapter is ligated to the 3′ end of the one or more of the first complementary strands of the target nucleic acids. In some embodiments, releasing the target nucleic acids from the surface is achieved by changing a condition. In further embodiments, the condition is temperature, pH, formamide concentration, or a combination thereof. In various embodiments, the biological sample is a tissue sample. In various embodiments, the method does not comprise use of a spatial barcode.

To further illustrate the present disclosure, examples are given herein. It is to be understood that these examples are provided for illustrative purposes and are not to be construed as limiting the scope of the present disclosure.

### EXAMPLES

### Example 1

Methods of spatially capturing target analytes (e.g., target nucleic acids) of a biological sample (e.g., tissue sample) are provided herein. The methods of the disclosure advantageously use the same surface (e.g., flow cell) for both capture of target analytes and sequencing readout.

A schematic workflow of a method of the disclosure is shown in FIG. 3. FIG. 3 shows a spatial workflow in which a tissue section is placed on a flow cell (FC) surface, wherein the FC surface comprises a plurality of capture oligonucleotides (comprising, for example, polyT oligonucleotides as shown in FIG. 3 and a clustering primer). RNA from the tissue section is captured by the capture oligonucleotide (probe) on a specific location on the FC. The tissue section is removed from the surface and the capture nucleotide sequence of the capture oligonucleotides is extended to form first complementary strands of the RNA. Next, the RNA is released from the surface by, e.g., dehybridization, and a clustering adapter is ligated to one or more of the first complementary strands. The clustering adapter comprises a binding site for a second clustering primer and a sequencing primer binding site. Next, the second clustering primer is hybridized to the binding site for the second clustering primer and is used to perform clustering (signal amplification) of the complementary strands. A sequencing primer is hybridized to the sequencing primer binding site and sequencing by synthesis is performed to read the sequences of the captured strands on the surface.

Another schematic workflow of a method of the disclosure is shown in FIG. 4. FIG. 4 shows a spatial workflow in which a tissue section is placed on a flow cell (FC) surface, wherein the FC surface comprises a plurality of first clustering primers (for example, P7 primers as shown in FIG. 4), some of which comprise a capture oligonucleotide (for example, a polyT oligonucleotide as shown in FIG. 4) attached thereto. RNA from the tissue section is captured by the capture oligonucleotide (probe) at a specific location on the FC. The tissue section is removed from the surface and the capture nucleotide sequence of the capture oligonucleotides is extended to form first complementary strands of the RNA. Next, the RNA is released from the surface by, e.g., dehybridization. Next, a random pool of clustering adapters is ligated to one or more or all of the first complementary strands. The clustering adapters each comprise (i) a binding site for a second clustering primer and (ii) a unique sequencing primer binding site (e.g., SBS3′, SBS4′, and SBS5′ as depicted in FIG. 4). Next, a second clustering primer is hybridized to the binding site for the second clustering primer of one or more or all of the first complementary strands of the RNA, and extension of the second clustering primer is performed. Then the complex between primers and recombinase interrogates double stranded DNA seeking a homologous sequence and promotes strand invasion and extension by the surface primer at the cognate site, thereby resulting in clustering (signal amplification) of the first complementary strands. Next, a specific sequencing primer is hybridized to its unique complementary sequencing primer binding site and sequencing by synthesis is performed to read the sequences of the first complementary strands on the surface. The sequencing by synthesis product is then dehybridized, and a different specific sequencing primer is hybridized to its unique complementary sequencing primer binding site and sequencing by synthesis is performed to read the sequences of the first complementary strands on the surface. Dehybridization of the sequencing by synthesis product, hybridization of specific sequencing primers, and sequencing by synthesis is repeated as desired.

Another schematic workflow of a method of the disclosure is shown in FIG. 5. Similar to FIG. 4, FIG. 5 shows a spatial workflow in which a tissue section is placed on a flow cell (FC) surface, wherein the FC surface comprises a plurality of first clustering primers (for example, P7 primers as shown in FIG. 5), some of which comprise a capture oligonucleotide (for example, a polyT oligonucleotide as shown in FIG. 5) attached thereto. RNA from the tissue section is captured by the capture oligonucleotide (probe) at a specific location on the FC. The tissue section is removed from the surface and the capture nucleotide sequence of the capture oligonucleotides is extended to form first complementary strands of the RNA. A specific enzyme used for the extension adds two or more random nucleotide bases to the 3′ end of the first complementary strands. Next, the RNA is released from the surface by, e.g., dehybridization. Next, a pool of oligonucleotides complementary to clustering adapters is hybridized to the two or more random bases at the 3′ ends of the first complementary strands of the RNA. The first complementary strands of the RNA are then extended to add clustering adapters. Each clustering adapter comprises (i) a binding site for a second clustering primer and (ii) a unique sequencing primer binding site (e.g., SBS3′, SBS4′, and SBS5′ as depicted in FIG. 5). Next, a second clustering primer is hybridized to the binding site for the second clustering primer of one or more or all of the first complementary strands of the RNA, and extension of the second clustering primer is performed. Then the complex between primers and recombinase interrogates double stranded DNA seeking a homologous sequence and promotes strand invasion and extension by the surface primer at the cognate site, thereby resulting in clustering (signal amplification) of the first complementary strands. Next, a specific sequencing primer is hybridized to its unique complementary sequencing primer binding site and sequencing by synthesis is performed to read the sequences of the first complementary strands on the surface. The sequencing by synthesis product is then dehybridized, and a different specific sequencing primer is hybridized to its unique complementary sequencing primer binding site and sequencing by synthesis is performed to read the sequences of the first complementary strands on the surface. Dehybridization of the sequencing by synthesis product, hybridization of specific sequencing primers, and sequencing by synthesis is repeated as desired.

1. A method for sequencing nucleic acids, comprising:
(a) providing:
(i) a plurality of first clustering primers immobilized on a surface, wherein each first clustering primer in the plurality of first clustering primers comprises a first clustering primer sequence; and
(ii) a plurality of capture oligonucleotides immobilized on the surface, wherein each capture oligonucleotide in the plurality of capture oligonucleotides comprises (1) the first clustering primer sequence that is immobilized on the surface and (2) a capture nucleotide sequence that is configured to bind to target nucleic acids of a biological sample;
(b) contacting the biological sample with the surface, the contacting resulting in hybridization of the target nucleic acids of the biological sample to the capture nucleotide sequences of the plurality of capture oligonucleotides to form hybridized capture oligonucleotides, such that a position of one or more of the target nucleic acids can be correlated with a position in the biological sample;
(c) removing the biological sample from the surface;
(d) extending the capture nucleotide sequence of the hybridized capture oligonucleotides to form first complementary strands of the target nucleic acids;
(e) releasing the target nucleic acids from the surface;
(f) ligating an adapter to one or more of the first complementary strands of the target nucleic acids to form ligated first complementary strands, wherein the adapter comprises a binding site for a second clustering primer and a binding site for a sequencing primer; and
(g) hybridizing a plurality of second clustering primers to the binding site for the second clustering primer on the ligated first complementary strands to generate hybridized second clustering primers and extending the hybridized second clustering primers to produce second complementary strands that comprise sequences of the target nucleic acids, or portions thereof;
(h) separating one or more of the ligated first complementary strands from the second complementary strands to generate separated second complementary strands and hybridizing the separated second complementary strands to a further first clustering primer of the plurality of first clustering primers to generate hybridized first clustering primers;
(i) hybridizing further second clustering primers of the plurality of second clustering primers to the binding site for the second clustering primer on the ligated first complementary strands;
(j) extending (1) one or more of the hybridized first clustering primers to generate additional first complementary strands, and (2) the further second clustering primers of the plurality of second clustering primers to generate additional second complementary strands;
(k) repeating steps (h) to (j) to produce one or more clusters of first complementary strands and second complementary strands;
(l) removing the second complementary strands from the surface and hybridizing a plurality of sequencing primers to the binding site for the sequencing primer on the one or more clusters of first complementary strands and sequencing the one or more clusters of first complementary strands to determine a sequence of the one or more clusters of first complementary strands.

2. (canceled)

3. The method of claim 1, wherein at least two of the ligated first complementary strands comprise different sequences for the sequencing primer binding sites.

4. The method of claim 3, wherein the one or more clusters of first complementary strands comprises a plurality of clusters of first complementary strands, and sequencing comprises:
(1) hybridizing a first plurality of sequencing primers to a first cluster of first complementary strands having a binding site for the first plurality of sequencing primers, and sequencing the first cluster of first complementary strands;
(2) removing the first plurality of sequencing primers from the surface;
(3) hybridizing a second plurality of sequencing primers to a second cluster of first complementary strands having a binding site for the second plurality of sequencing primers, and sequencing the second cluster of first complementary strands; and optionally
(4) repeating steps (1) to (3) with one or more additional pluralities of sequencing primers.

5. A method for sequencing nucleic acids, comprising:
(a) providing (i) a plurality of first clustering primers immobilized on a surface, wherein each first clustering primer in the plurality of first clustering primers comprises a first clustering primer sequence and (ii) a plurality of capture oligonucleotides immobilized on the surface, wherein each capture oligonucleotide in the plurality of capture oligonucleotides comprises (1) the first clustering primer sequence that is immobilized on the surface and (2) a capture nucleotide sequence that is configured to bind to target nucleic acids of a biological sample;
(b) contacting the biological sample with the surface, the contacting resulting in hybridization of the target nucleic acids of the biological sample to the capture nucleotide sequence of the plurality of capture oligonucleotides to form hybridized capture oligonucleotides, such that a position of one or more of the target nucleic acids can be correlated with a position in the biological sample;
(c) removing the biological sample from the surface;
(d) extending the capture nucleotide sequence of the hybridized capture oligonucleotides to form first complementary strands of the target nucleic acids, wherein the extending comprises addition of a plurality of non-templated nucleotides to the end of the first complementary strands;
(e) hybridizing a plurality of template switching oligonucleotides to the plurality of non-templated nucleotides of the first complementary strands such that each of the plurality of template switching oligonucleotides is positioned at the terminus of the target nucleic acids that is distal to the surface, wherein each oligonucleotide in the plurality of template switching oligonucleotides comprises (1) a nucleotide sequence that binds to the plurality of non-templated nucleotides; (2) a sequencing primer sequence; and (3) a second clustering primer sequence;
(f) extending the plurality of non-templated nucleotides on the first complementary strands using the template switching oligonucleotides as template, thereby generating a binding site for a second clustering primer and a binding site for a sequencing primer on the first complementary strands;
(g) releasing the target nucleic acids from the surface;
(h) hybridizing a plurality of second clustering primers to the binding site for the second clustering primer on the first complementary strands to generate hybridized second clustering primers and extending the hybridized second clustering primers to produce second complementary strands that comprise sequences of the target nucleic acids, or portions thereof;
(i) separating one or more of the first complementary strands from the second complementary strands to generate separated second complementary strands and hybridizing the separated second complementary strands to a further first clustering primer of the plurality of first clustering primers to generate hybridized first clustering primers;
(j) hybridizing further second clustering primers of the plurality of second clustering primers to the binding site for the second clustering primer on the first complementary strands;
(k) extending (1) one or more of the hybridized first clustering primers to generate additional first complementary strands, and (2) the further second clustering primers of the plurality of second clustering primers to generate additional second complementary strands;
(l) repeating steps (i) to (k) to produce one or more clusters of first complementary strands and second complementary strands;
(m) removing the second complementary strands from the surface and hybridizing a plurality of sequencing primers to the binding site for the sequencing primer on the one or more clusters of first complementary strands and sequencing the one or more clusters of first complementary strands to determine a sequence of the one or more clusters of first complementary strands.

6. (canceled)

7. The method of claim 5, wherein at least two of the plurality of template switching oligonucleotides comprise sequencing primer sequences that are different.

8. The method of claim 7, wherein the one or more clusters of first complementary strands comprises a plurality of clusters of first complementary strands, and sequencing comprises:
(1) hybridizing a first plurality of sequencing primers to a first cluster of first complementary strands having a binding site for the first plurality of sequencing primers, and sequencing the first cluster of first complementary strands;
(2) removing the first plurality of sequencing primers from the surface;
(3) hybridizing a second plurality of sequencing primers to a second cluster of first complementary strands having a binding site for the second plurality of sequencing primers, and sequencing the second cluster of first complementary strands; and optionally
(4) repeating steps (1) to (3) with one or more additional pluralities of sequencing primers.

9. The method of claim 1, further comprising staining the biological sample after the contacting but before removing the biological sample from the surface.

10. (canceled)

11. (canceled)

12. The method of claim 1, further comprising correlating the sequence of the one or more clusters of first complementary strands to a position of the target nucleic acids in the biological sample.

13. (canceled)

14. (canceled)

15. (canceled)

16. (canceled)

17. The method of claim 4, wherein:
(a) each sequencing primer in the first plurality of sequencing primers comprises the same nucleotide sequence;
(b) each sequencing primer in the second plurality of sequencing primers comprises the same nucleotide sequence;
(c) each sequencing primer in each of the one or more additional pluralities of sequencing primers comprises the same nucleotide sequence; and
(d) one or more of: the first plurality of sequencing primers, the second plurality of sequencing primers, and each of the one or more additional pluralities of sequencing primers comprise different nucleotide sequences.

18. A method for obtaining spatial information about target nucleic acids of a biological sample, comprising:
(a) providing (i) a plurality of first clustering primers immobilized on a surface, wherein each first clustering primer in the plurality of first clustering primers comprises a first clustering primer sequence and (ii) a plurality of capture oligonucleotides immobilized on the surface, wherein each capture oligonucleotide in the plurality of capture oligonucleotides comprises (1) a first clustering primer sequence that is immobilized on the surface and (2) a capture nucleotide sequence that is configured to bind to the target nucleic acids of the biological sample;
(b) contacting the biological sample with the surface, the contacting resulting in hybridization of the target nucleic acids of the biological sample to the capture nucleotide sequences of the plurality of capture oligonucleotides to form hybridized capture oligonucleotides, such that a position of one or more of the target nucleic acids can be correlated with a position in the biological sample;
(c) removing the biological sample from the surface;
(d) extending the capture nucleotide sequence of the hybridized capture oligonucleotides to form first complementary strands of the target nucleic acids;
(e) releasing the target nucleic acids from the surface;
(f) ligating an adapter to one or more of the first complementary strands of the target nucleic acids to form ligated first complementary strands, wherein the adapter comprises a binding site for a second clustering primer and a binding site for a sequencing primer;
(g) hybridizing a plurality of second clustering primers to the binding site for the second clustering primer on the first complementary strands to generate hybridized second clustering primers and extending the hybridized second clustering primers to produce second complementary strands that comprise sequences of the target nucleic acids, or portions thereof;
(h) separating one or more of the ligated first complementary strands from the second complementary strands to generate separated second complementary strands and hybridizing the separated second complementary strands to a further first clustering primer of the plurality of first clustering primers to generate hybridized first clustering primers;
(i) hybridizing further second clustering primers of the plurality of second clustering primers to the binding site for the second clustering primer on the ligated first complementary strands;
(j) extending (1) one or more of the hybridized first clustering primers to generate additional first complementary strands, and (2) the further second clustering primers of the plurality of second clustering primers to generate additional second complementary strands;
(k) repeating steps (h) to (j) to produce one or more clusters of first complementary strands and second complementary strands;
(l) removing the second complementary strands from the surface and hybridizing a plurality of sequencing primers to the binding site for the sequencing primer on the one or more clusters of first complementary strands and sequencing the one or more clusters of first complementary strands to determine a sequence of the one or more clusters of first complementary strands; and
(m) correlating the sequence of the one or more clusters of first complementary strands to a position of the target nucleic acids in the biological sample.

19. (canceled)

20. The method of claim 18, wherein the binding site for the sequencing primer of at least two of the ligated first complementary strands comprises a different nucleotide sequence.

21. The method of claim 20, wherein the one or more clusters of first complementary strands comprises a plurality of clusters of first complementary strands, and sequencing comprises:
(1) hybridizing a first plurality of sequencing primers to a first cluster of first complementary strands having a binding site for the first plurality of sequencing primers, and sequencing the first cluster of first complementary strands;
(2) removing the first plurality of sequencing primers from the surface;
(3) hybridizing a second plurality of sequencing primers to a second cluster of first complementary strands having a binding site for the second plurality of sequencing primers, and sequencing the second cluster of first complementary strands; and optionally
(4) repeating steps (1) to (3) with one or more additional pluralities of sequencing primers.

22. A method for obtaining spatial information about target nucleic acids of a biological sample, comprising:
(a) providing (i) a plurality of first clustering primers immobilized on a surface, wherein each first clustering primer in the plurality of first clustering primers comprises a first clustering primer sequence and (ii) a plurality of capture oligonucleotides immobilized on the surface, wherein each capture oligonucleotide in the plurality of capture oligonucleotides comprises (1) the first clustering primer sequence that is immobilized on the surface and (2) a capture nucleotide sequence that is configured to bind to the target nucleic acids of the biological sample;
(b) contacting the biological sample with the surface, the contacting resulting in hybridization of the target nucleic acids of the biological sample to the capture nucleotide sequence of the plurality of capture oligonucleotides to form hybridized capture oligonucleotides, such that a position of one or more of the target nucleic acids can be correlated with a position in the biological sample;
(c) removing the biological sample from the surface;
(d) extending the capture nucleotide sequence of the hybridized capture oligonucleotides to form first complementary strands of the target nucleic acids, wherein the extending comprises addition of a plurality of non-templated nucleotides to the end of the first complementary strands;
(e) hybridizing a plurality of template switching oligonucleotides to the plurality of non-templated nucleotides of the first complementary strands such that each of the plurality of template switching oligonucleotides is positioned at the terminus of the target nucleic acids that is distal to the surface, wherein each oligonucleotide in the plurality of template switching oligonucleotides comprises (1) a nucleotide sequence that binds to the plurality of non-templated nucleotides; (2) a sequencing primer sequence; and (3) a second clustering primer sequence;
(f) extending the plurality of non-templated nucleotides on the first complementary strands using the template switching oligonucleotides as template, thereby generating a binding site for a second clustering primer and a binding site for a sequencing primer on the first complementary strands;
(g) releasing the target nucleic acids from the surface;
(h) hybridizing a plurality of second clustering primers to the binding site for the second clustering primer on the first complementary strands to generate hybridized second clustering primers and extending the hybridized second clustering primers to produce second complementary strands that comprise sequences of the target nucleic acids, or portions thereof;
(i) separating one or more of the first complementary strands from the second complementary strands to generate separated second complementary strands and hybridizing the separated second complementary strands to a further first clustering primer of the plurality of first clustering primers to generate hybridized first clustering primers;
(j) hybridizing further second clustering primers of the plurality of second clustering primers to the binding site for the second clustering primer on the first complementary strands;
(k) extending (1) one or more of the hybridized first clustering primers to generate additional first complementary strands, and (2) the further second clustering primers of the plurality of second clustering primers to generate additional second complementary strands;
(l) repeating steps (i) to (k) to produce one or more clusters of first complementary strands and second complementary strands;
(m) removing the second complementary strands from the surface and hybridizing a plurality of sequencing primers to the binding site for the sequencing primer on the one or more clusters of first complementary strands and sequencing the one or more clusters of first complementary strands to determine a sequence of the one or more clusters of first complementary strands; and
(n) correlating the sequence of the clusters of first complementary strands to a position of the target nucleic acids in the biological sample.

23. (canceled)

24. The method of claim 22, wherein at least two of the plurality of template switching oligonucleotides comprise sequencing primer sequences that are different.

25. The method of claim 24, wherein the one or more clusters of first complementary strands comprises a plurality of clusters of first complementary strands, and sequencing comprises:
(1) hybridizing a first plurality of sequencing primers to a first cluster of first complementary strands having a binding site for the first plurality of sequencing primers, and sequencing the first cluster of first complementary strands;
(2) removing the first plurality of sequencing primers from the surface;
(3) hybridizing a second plurality of sequencing primers to a second cluster of first complementary strands having a binding site for the second plurality of sequencing primers, and sequencing the second cluster of first complementary strands; and optionally
(4) repeating steps (1) to (3) with one or more additional pluralities of sequencing primers.

26. The method of claim 18, further comprising staining the biological sample after the contacting but before removing the biological sample from the surface.

27. (canceled)

28. (canceled)

29. (canceled)

30. (canceled)

31. (canceled)

32. A method for obtaining spatial information about target nucleic acids of a biological sample, comprising:
(a) providing:
(I) a plurality of capture oligonucleotides immobilized on a surface, wherein each capture oligonucleotide in the plurality of capture oligonucleotides comprises (i) a first clustering primer sequence that is immobilized on the surface; (ii) a sequencing primer sequence; and (iii) a capture nucleotide sequence that is configured to bind to the target nucleic acids of the biological sample;
(II) a plurality of first clustering primers immobilized on the surface, wherein each first clustering primer in the plurality of first clustering primers comprises the first clustering primer sequence; and
(III) a plurality of dormant second clustering primers immobilized on the surface, wherein each dormant second clustering primer in the plurality of dormant second clustering primers is blocked at the 3′ end;
(b) contacting the biological sample with the surface, the contacting resulting in hybridization of the target nucleic acids of the biological sample to the capture nucleotide sequence of the plurality of capture oligonucleotides to form hybridized capture oligonucleotides, such that a position of one or more of the target nucleic acids can be correlated with a position in the biological sample;
(c) removing the biological sample from the surface;
(d) extending the capture nucleotide sequence of the hybridized capture oligonucleotides to form first complementary strands of the target nucleic acids;
(e) releasing the target nucleic acids from the surface;
(f) ligating an adapter to one or more of the first complementary strands of the target nucleic acids to form ligated first complementary strands, wherein the adapter comprises a binding site for a third clustering primer and a binding site for a sequencing primer; and
(g) hybridizing a plurality of third clustering primers to the binding site for the third clustering primer on the ligated first complementary strands to generate hybridized third clustering primers and extending the hybridized third clustering primers to produce second complementary strands that comprise sequences of the target nucleic acids, or portions thereof;
(h) separating one or more of the second complementary strands and hybridizing the one or more of the second complementary strands to the plurality of first clustering primers immobilized on the surface;
(i) providing a plurality of primers that hybridizes to the binding site for the third clustering primer of the ligated first complementary strands;
(j) extending the third clustering primer and the primer of step (i) to generate additional complementary strands;
(k) repeating steps (h) to (j) to produce a cluster of complementary strands;
(l) removing strands not immobilized on the surface;
(m) carrying out a first sequencing read to determine a sequence of a region of immobilized strands;
(n) removing sequencing products;
(o) removing the blocking group from the plurality of dormant second clustering primers to allow hybridization of the 3′ end of strands immobilized on the surface to the dormant second clustering primers;
(p) extending the dormant second clustering primers using strands immobilized on the surface as a template;
(q) removing the first sequencing read strands;
(r) carrying out a second sequencing read to determine a sequence of immobilized strands; and correlating the second sequencing reads to the position of the target nucleic acids in the biological sample, thereby obtaining spatial information about the target nucleic acids of the biological sample.

33. The method of claim 1, wherein the surface is a planar surface or a bead surface.

34. The method of claim 33, wherein the planar surface is a flow cell surface.

35. The method of claim 1, wherein the ratio of capture oligonucleotides to clustering oligonucleotides on the surface is about 1:10.

36. (canceled)

37. The method of claim 1, wherein the plurality of capture oligonucleotides comprises multiple, different capture nucleotide sequences.

38. The method of claim 37, wherein the multiple, different capture nucleotide sequences comprise one or more gene-specific capture sequences, one or more universal capture sequences, or a combination thereof.

39. The method of claim 1, wherein the capture nucleotide sequence is a poly-T sequence, a poly-A sequence, a gene-specific capture sequence, or a universal capture sequence.

40. The method of claim 38, wherein the universal capture sequence is a random nucleotide sequence or a non-self complementary semi-random sequence.

41. The method of claim 1, wherein the target nucleic acids are mRNA, gDNA, RNA, tRNA, or a combination thereof.

42. (canceled)

43. (canceled)

44. The method of claim 1, wherein the target nucleic acids are polyadenylated prior to hybridization of the target nucleic acids to the capture nucleotide sequences.

45. The method of claim 44, wherein the target nucleic acids are polyadenylated using a poly(A) polymerase.

46. The method of claim 44, wherein the target nucleic acids are polyadenylated using chemical ligation or enzymatic ligation.

47. The method of claim 1, wherein the ligating is achieved using chemical ligation or enzymatic ligation.

48. The method of claim 1, wherein steps (h) to (j) are repeated through multiple cycles in the presence of a recombinase.

49. The method of claim 5, wherein steps (i) to (k) are repeated through multiple cycles in the presence of a recombinase.

50. The method of claim 1, wherein the adapter is ligated to the 3′ end of the one or more of the first complementary strands of the target nucleic acids.

51. The method of claim 1, wherein releasing the target nucleic acids from the surface is achieved by changing a condition.

52. The method of claim 51, wherein the condition is temperature, pH, formamide concentration, or a combination thereof.

53. The method of claim 1, wherein the biological sample is a tissue sample.

54. The method of claim 1, wherein the method does not comprise use of a spatial barcode.


SEQUENCE LISTING (8 sequences)

SEQ ID NO: 1 - DNA, 20 bp, synthetic construct
  source 1..20: mol_type = other DNA; note = P5 sequence (short)
aatgatacggcgaccaccga

SEQ ID NO: 2 - DNA, 29 bp, synthetic construct
  source 1..29: mol_type = other DNA; note = P5 sequence (long)
aatgatacggcgaccaccgagatctacac

SEQ ID NO: 3 - DNA, 20 bp, synthetic construct
  source 1..20: mol_type = other DNA; note = P7 sequence (short)
caagcagaagacggcatacg

SEQ ID NO: 4 - DNA, 24 bp, synthetic construct
  source 1..24: mol_type = other DNA; note = P7 sequence (long)
caagcagaagacggcatacgagat

SEQ ID NO: 5 - DNA, 20 bp, synthetic construct
  source 1..20: mol_type = other DNA; note = P5 sequence
tcggtggtcgccgtatcatt

SEQ ID NO: 6 - DNA, 29 bp, synthetic construct
  source 1..29: mol_type = other DNA; note = P5 sequence
gtgtagatctcggtggtcgccgtatcatt

SEQ ID NO: 7 - DNA, 20 bp, synthetic construct
  source 1..20: mol_type = other DNA; note = P7 sequence
cgtatgccgtcttctgcttg

SEQ ID NO: 8 - DNA, 24 bp, synthetic construct
  source 1..24: mol_type = other DNA; note = P7 sequence
atctcgtatgccgtcttctgcttg